SEQUENCE LISTING (291 sequences)

SEQ ID NO: 1 - protein, 6 aa, Artificial Sequence
  Peptide conjugate
FCIGRL

SEQ ID NO: 2 - protein, 6 aa, Artificial Sequence
  Peptide conjugate
ACIGRL

SEQ ID NO: 3 - protein, 6 aa, Artificial Sequence
  Peptide conjugate
FAIGRL

SEQ ID NO: 4 - protein, 6 aa, Artificial Sequence
  Peptide conjugate
FCAGRL

SEQ ID NO: 5 - protein, 6 aa, Artificial Sequence
  Peptide conjugate
FCIARL

SEQ ID NO: 6 - protein, 6 aa, Artificial Sequence
  Peptide conjugate
FCIGAL

SEQ ID NO: 7 - protein, 6 aa, Artificial Sequence
  Peptide conjugate
FCIGRA

SEQ ID NO: 8 - protein, 5 aa, Artificial Sequence
  Peptide conjugate
CIGRL

SEQ ID NO: 9 - protein, 4 aa, Artificial Sequence
  Peptide conjugate
IGRL

SEQ ID NO: 10 - protein, 5 aa, Artificial Sequence
  Peptide conjugate
FCIGR

SEQ ID NO: 11 - protein, 4 aa, Artificial Sequence
  Peptide conjugate
FCIG

SEQ ID NO: 12 - protein, 6 aa, Artificial Sequence
  Peptide conjugate
  misc_feature (1)..(1): Phe may be D-Phe
FCIGRL

SEQ ID NO: 13 - protein, 6 aa, Artificial Sequence
  Peptide conjugate
  misc_feature (2)..(2): Cys may be D-Cys
FCIGRL

SEQ ID NO: 14 - protein, 6 aa, Artificial Sequence
  Peptide conjugate
  misc_feature (3)..(3): Ile may be D-Ile
FCIGRL

SEQ ID NO: 15 - protein, 6 aa, Artificial Sequence
  Peptide conjugate
  misc_feature (5)..(5): Arg may be D-Arg
FCIGRL

SEQ ID NO: 16 - protein, 6 aa, Artificial Sequence
  Peptide conjugate
  misc_feature (6)..(6): Leu may be D-Leu
FCIGRL

SEQ ID NO: 17 - protein, 6 aa, Artificial Sequence
  Peptide conjugate
  misc_feature (1)..(1): Leu may be D-Leu
  misc_feature (2)..(2): Arg may be D-Arg
  misc_feature (4)..(4): Ile may be D-Ile
  misc_feature (5)..(5): Cys may be D-Cys
  misc_feature (6)..(6): Phe may be D-Phe
LRGICF

SEQ ID NO: 18 - protein, 6 aa, Artificial Sequence
  Peptide conjugate
  misc_feature (1)..(1): Leu may be D-Leu
  misc_feature (2)..(2): Arg may be D-Arg
  misc_feature (4)..(4): Ile may be D-Ile
  misc_feature (5)..(5): Cys may be D-Cys
LRGICF

SEQ ID NO: 19 - protein, 6 aa, Artificial Sequence
  Peptide conjugate
  misc_feature (1)..(1): Leu may be D-Leu
  misc_feature (2)..(2): Arg may be D-Arg
  misc_feature (4)..(4): Ile may be D-Ile
  misc_feature (6)..(6): Phe may be D-Phe
LRGICF

SEQ ID NO: 20 - protein, 6 aa, Artificial Sequence
  Peptide conjugate
  misc_feature (1)..(1): Leu may be D-Leu
  misc_feature (2)..(2): Arg may be D-Arg
  misc_feature (5)..(5): Cys may be D-Cys
  misc_feature (6)..(6): Phe may be D-Phe
LRGICF

SEQ ID NO: 21 - protein, 6 aa, Artificial Sequence
  Peptide conjugate
  misc_feature (1)..(1): Leu may be D-Leu
  misc_feature (4)..(4): Ile may be D-Ile
  misc_feature (5)..(5): Cys may be D-Cys
  misc_feature (6)..(6): Phe may be D-Phe
LRGICF

SEQ ID NO: 22 - protein, 6 aa, Artificial Sequence
  Peptide conjugate
  misc_feature (2)..(2): Arg may be D-Arg
  misc_feature (4)..(4): Ile may be D-Ile
  misc_feature (5)..(5): Cys may be D-Cys
  misc_feature (6)..(6): Phe may be D-Phe
LRGICF

SEQ ID NO: 23 - protein, 6 aa, Artificial Sequence
  Peptide conjugate
LRGICF

SEQ ID NO: 24 - protein, 6 aa, Artificial Sequence
  Peptide conjugate
  misc_feature (1)..(1): Leu may be D-Leu
LRGICF

SEQ ID NO: 25 - protein, 6 aa, Artificial Sequence
  Peptide conjugate
  misc_feature (2)..(2): Arg may be D-Arg
LRGICF

SEQ ID NO: 26 - protein, 6 aa, Artificial Sequence
  Peptide conjugate
  misc_feature (4)..(4): Ile may be D-Ile
LRGICF

SEQ ID NO: 27 - protein, 6 aa, Artificial Sequence
  Peptide conjugate
  misc_feature (5)..(5): Cys may be D-Cys
LRGICF

SEQ ID NO: 28 - protein, 6 aa, Artificial Sequence
  Peptide conjugate
  misc_feature (6)..(6): Phe may be D-Phe
LRGICF

SEQ ID NO: 29 - protein, 6 aa, Artificial Sequence
  Peptide conjugate
  misc_feature (1)..(1): Phe may be D-Phe
  misc_feature (2)..(2): Cys may be D-Cys
  misc_feature (4)..(4): Ile may be D-Ile
  misc_feature (5)..(5): Arg may be D-Arg
  misc_feature (6)..(6): Leu may be D-Leu
FCIGRL

SEQ ID NO: 30 - protein, 6 aa, Artificial Sequence
  Peptide conjugate
  misc_feature (1)..(1): Phe may be D-Phe
  misc_feature (2)..(2): Cys may be D-Cys
  misc_feature (4)..(4): Ile may be D-Ile
  misc_feature (5)..(5): Arg may be D-Arg
FCIGRL

SEQ ID NO: 31 - protein, 6 aa, Artificial Sequence
  Peptide conjugate
  misc_feature (1)..(1): Phe may be D-Phe
  misc_feature (2)..(2): Cys may be D-Cys
  misc_feature (4)..(4): Ile may be D-Ile
  misc_feature (6)..(6): Leu may be D-Leu
FCIGRL

SEQ ID NO: 32 - protein, 6 aa, Artificial Sequence
  Peptide conjugate
  misc_feature (1)..(1): Phe may be D-Phe
  misc_feature (2)..(2): Cys may be D-Cys
  misc_feature (5)..(5): Arg may be D-Arg
  misc_feature (6)..(6): Leu may be D-Leu
FCIGRL

SEQ ID NO: 33 - protein, 6 aa, Artificial Sequence
  Peptide conjugate
  misc_feature (1)..(1): Phe may be D-Phe
  misc_feature (4)..(4): Ile may be D-Ile
  misc_feature (5)..(5): Arg may be D-Arg
  misc_feature (6)..(6): Leu may be D-Leu
FCIGRL

SEQ ID NO: 34 - protein, 6 aa, Artificial Sequence
  Peptide conjugate
  misc_feature (2)..(2): Cys may be D-Cys
  misc_feature (4)..(4): Ile may be D-Ile
  misc_feature (5)..(5): Arg may be D-Arg
  misc_feature (6)..(6): Leu may be D-Leu
FCIGRL

SEQ ID NO: 35 - protein, 6 aa, Artificial Sequence
  Peptide conjugate
  misc_feature (2)..(2): Cys may be D-Cys
  misc_feature (5)..(5): Arg may be D-Arg
FCIGRL

SEQ ID NO: 36 - protein, 6 aa, Artificial Sequence
  Peptide conjugate
  misc_feature (1)..(1): Leu may be D-Leu
LRGICF

SEQ ID NO: 37 - protein, 6 aa, Artificial Sequence
  Peptide conjugate
  misc_feature (1)..(1): Leu may be D-Leu
  misc_feature (2)..(2): Arg may be D-Arg
LRGICF

SEQ ID NO: 38 - protein, 6 aa, Artificial Sequence
  Peptide conjugate
  misc_feature (1)..(1): Leu may be D-Leu
  misc_feature (2)..(2): Arg may be D-Arg
  misc_feature (6)..(6): Phe may be D-Phe
LRGICF

SEQ ID NO: 39 - protein, 6 aa, Artificial Sequence
  Peptide conjugate
  misc_feature (1)..(1): Leu may be D-Leu
  misc_feature (2)..(2): Arg may be D-Arg
  misc_feature (5)..(5): Cys may be D-Cys
LRGICF

SEQ ID NO: 40 - protein, 6 aa, Artificial Sequence
  Peptide conjugate
  misc_feature (6)..(6): Ser may be carboxylated
FCAGMS

SEQ ID NO: 41 - protein, 6 aa, Artificial Sequence
  Peptide conjugate
  misc_feature (6)..(6): Ser may be carboxylated
FCVGMS

SEQ ID NO: 42 - protein, 6 aa, Artificial Sequence
  Peptide conjugate
  misc_feature (1)..(1): May be protiated
  misc_feature (6)..(6): May be hydroxylated
PCIGRL

SEQ ID NO: 43 - protein, 6 aa, Artificial Sequence
  Peptide conjugate
  misc_feature (1)..(1): May be protiated
  misc_feature (6)..(6): May be hydroxylated
QCIGRL

SEQ ID NO: 44 - protein, 6 aa, Artificial Sequence
  Peptide conjugate
  misc_feature (1)..(1): May be protiated
  misc_feature (6)..(6): May be hydroxylated
GCIGRL

SEQ ID NO: 45 - protein, 6 aa, Artificial Sequence
  Peptide conjugate
  misc_feature (1)..(1): May be protiated
  misc_feature (6)..(6): May be hydroxylated
TCIGRL

SEQ ID NO: 46 - protein, 6 aa, Artificial Sequence
  Peptide conjugate
  misc_feature (1)..(1): May be protiated
  misc_feature (6)..(6): May be hydroxylated
SCIGRL

SEQ ID NO: 47 - protein, 6 aa, Artificial Sequence
  Peptide conjugate
  misc_feature (1)..(1): May be protiated
  misc_feature (6)..(6): May be hydroxylated
NCIGRL

SEQ ID NO: 48 - protein, 6 aa, Artificial Sequence
  Peptide conjugate
  misc_feature (1)..(1): May be protiated
  misc_feature (6)..(6): May be hydroxylated
RCIGRL

SEQ ID NO: 49 - protein, 6 aa, Artificial Sequence
  Peptide conjugate
  misc_feature (1)..(1): May be protiated
  misc_feature (6)..(6): May be hydroxylated
VCIGRL

SEQ ID NO: 50 - protein, 6 aa, Artificial Sequence
  Peptide conjugate
  misc_feature (1)..(1): May be protiated
  misc_feature (6)..(6): May be hydroxylated
FCIGRG

SEQ ID NO: 51 - protein, 6 aa, Artificial Sequence
  Peptide conjugate
  misc_feature (1)..(1): May be protiated
  misc_feature (1)..(1): Ala may be D-Ala
  misc_feature (6)..(6): May be hydroxylated
ACIGRG

SEQ ID NO: 52 - protein, 6 aa, Artificial Sequence
  Peptide conjugate
  misc_feature (1)..(1): May be protiated
  misc_feature (6)..(6): May be hydroxylated
ACIGRG

SEQ ID NO: 53 - protein, 6 aa, Artificial Sequence
  Peptide conjugate
  misc_feature (1)..(1): May be protiated
  misc_feature (6)..(6): May be hydroxylated
FCIGRG

SEQ ID NO: 54 - protein, 6 aa, Artificial Sequence
  Peptide conjugate
  misc_feature (1)..(1): May be protiated
  misc_feature (1)..(1): Phe may be D-Phe
  misc_feature (6)..(6): May be hydroxylated
FCIGRG

SEQ ID NO: 55 - protein, 6 aa, Artificial Sequence
  Peptide conjugate
  misc_feature (1)..(1): May be protiated
  misc_feature (6)..(6): May be hydroxylated
FCIGRS

SEQ ID NO: 56 - protein, 6 aa, Artificial Sequence
  Peptide conjugate
  misc_feature (1)..(1): May be protiated
  misc_feature (6)..(6): May be hydroxylated
FCIGRQ

SEQ ID NO: 57 - protein, 6 aa, Artificial Sequence
  Peptide conjugate
  misc_feature (1)..(1): May be protiated
  misc_feature (6)..(6): May be hydroxylated
FCIGRK

SEQ ID NO: 58 - protein, 6 aa, Artificial Sequence
  Peptide conjugate
  misc_feature (1)..(1): May be protiated
  misc_feature (6)..(6): Ala may be D-Ala
  misc_feature (6)..(6): May be hydroxylated
FCIGRA

SEQ ID NO: 59 - protein, 6 aa, Artificial Sequence
  Peptide conjugate
  misc_feature (1)..(1): May be protiated
  misc_feature (6)..(6): May be hydroxylated
FCIGRI

SEQ ID NO: 60 - protein, 6 aa, Artificial Sequence
  Peptide conjugate
  misc_feature (1)..(1): May be protiated
  misc_feature (6)..(6): May be amidated
FCIGRG

SEQ ID NO: 61 - protein, 6 aa, Artificial Sequence
  Peptide conjugate
  misc_feature (1)..(1): May be protiated
  misc_feature (6)..(6): May be hydroxylated
FCIGRD

SEQ ID NO: 62 - protein, 6 aa, Artificial Sequence
  Peptide conjugate
  misc_feature (1)..(1): May be protiated
  misc_feature (6)..(6): May be hydroxylated
FCIGRE

SEQ ID NO: 63 - protein, 6 aa, Artificial Sequence
  Peptide conjugate
  misc_feature (1)..(1): May be protiated
  misc_feature (6)..(6): May be hydroxylated
FCIGRF

SEQ ID NO: 64 - protein, 6 aa, Artificial Sequence
  Peptide conjugate
  misc_feature (1)..(1): May be protiated
  misc_feature (6)..(6): May be hydroxylated
FCIGRN

SEQ ID NO: 65 - protein, 6 aa, Artificial Sequence
  Peptide conjugate
  misc_feature (1)..(1): May be protiated
  misc_feature (6)..(6): May be hydroxylated
FCIGRP

SEQ ID NO: 66 - protein, 6 aa, Artificial Sequence
  Peptide conjugate
  misc_feature (1)..(1): May be protiated
  misc_feature (6)..(6): May be hydroxylated
ECIGRL

SEQ ID NO: 67 - protein, 6 aa, Artificial Sequence
  Peptide conjugate
  misc_feature (1)..(1): May be protiated
  misc_feature (6)..(6): May be hydroxylated
DCIGRL

SEQ ID NO: 68 - protein, 6 aa, Artificial Sequence
  Peptide conjugate
  misc_feature (1)..(1): May be protiated
  misc_feature (6)..(6): May be hydroxylated
KCIGRL

SEQ ID NO: 69 - protein, 7 aa, Artificial Sequence
  Peptide conjugate
  misc_feature (1)..(1): May be protiated
  misc_feature (7)..(7): May be hydroxylated
FCIGRLC

SEQ ID NO: 70 - protein, 6 aa, Artificial Sequence
  Peptide conjugate
  misc_feature (1)..(1): May be protiated
  misc_feature (6)..(6): May be hydroxylated
PGPGRL

SEQ ID NO: 71 - protein, 6 aa, Artificial Sequence
  Peptide conjugate
  misc_feature (1)..(1): May be protiated
  misc_feature (6)..(6): May be hydroxylated
FCIPGP

SEQ ID NO: 72 - protein, 6 aa, Artificial Sequence
  Peptide conjugate
  misc_feature (1)..(1): May be protiated
  misc_feature (6)..(6): May be hydroxylated
FCLGRL

SEQ ID NO: 73 - protein, 6 aa, Artificial Sequence
  Peptide conjugate
  misc_feature (1)..(1): May be protiated
  misc_feature (6)..(6): May be hydroxylated
GCIGRG

SEQ ID NO: 74 - protein, 6 aa, Artificial Sequence
  Peptide conjugate
YCIGRL

SEQ ID NO: 75 - protein, 6 aa, Artificial Sequence
  Peptide conjugate
  misc_feature (1)..(1): May be acetylated
ACIGRL

SEQ ID NO: 76 - protein, 6 aa, Artificial Sequence
  Peptide conjugate
WCIGRL

SEQ ID NO: 77 - protein, 6 aa, Artificial Sequence
  Peptide conjugate
  misc_feature (1)..(1): May be acetylated
ACIGRS

SEQ ID NO: 78 - protein, 6 aa, Artificial Sequence
  Peptide conjugate
  misc_feature (1)..(1): May be acetylated
ACIGRA

SEQ ID NO: 79 - protein, 6 aa, Artificial Sequence
  Peptide conjugate
  misc_feature (1)..(1): May be acetylated
FCIGRF

SEQ ID NO: 80 - protein, 6 aa, Artificial Sequence
  Peptide conjugate
  misc_feature (6)..(6): May be amidated
SLIGRL

SEQ ID NO: 81 - protein, 4 aa, Artificial Sequence
  Peptide conjugate
FCAG

SEQ ID NO: 82 - protein, 4 aa, Artificial Sequence
  Peptide conjugate
FCGG

SEQ ID NO: 83 - protein, 7 aa, Artificial Sequence
  Peptide conjugate
GFCIGRL

SEQ ID NO: 84 - protein, 6 aa, Artificial Sequence
  Peptide conjugate
LRGGRL

SEQ ID NO: 85 - protein, 6 aa, Artificial Sequence
  Peptide conjugate
FCAGMS

SEQ ID NO: 86 - protein, 6 aa, Artificial Sequence
  Peptide conjugate
FCVGMS

SEQ ID NO: 87 - protein, 6 aa, Artificial Sequence
  Peptide conjugate
  misc_feature (1)..(1): May be acetylated
FLIGRL

SEQ ID NO: 88 - protein, 5 aa, Artificial Sequence
  Peptide conjugate
YIGSR

SEQ ID NO: 89 - protein, 6 aa, Artificial Sequence
  Peptide conjugate
  misc_feature (1)..(1): May be protiated
  misc_feature (1)..(1): Xaa may be sarcosine
  misc_feature (6)..(6): May be hydroxylated
XCIGRL

SEQ ID NO: 90 - protein, 6 aa, Artificial Sequence
  Peptide conjugate
  misc_feature (1)..(1): May be protiated
  misc_feature (1)..(1): Xaa may be L-beta-cyclohexyl-alanine
  misc_feature (6)..(6): May be hydroxylated
XCIGRL

SEQ ID NO: 91 - protein, 6 aa, Artificial Sequence
  Peptide conjugate
  misc_feature (1)..(1): May be protiated
  misc_feature (1)..(1): Xaa may be alpha-aminoisobutyric acid
  misc_feature (6)..(6): May be hydroxylated
XCIGRL

SEQ ID NO: 92 - protein, 7 aa, Artificial Sequence
  Peptide conjugate
  misc_feature (1)..(1): May be protiated
  misc_feature (1)..(1): Xaa may be L-tert-butylglycine
  misc_feature (6)..(6): May be hydroxylated
XGCIGRL

SEQ ID NO: 93 - protein, 6 aa, Artificial Sequence
  Peptide conjugate
  misc_feature (1)..(1): May be protiated
  misc_feature (6)..(6): May be hydroxylated
  misc_feature (1)..(1): Xaa may be norvaline
XCIGRL

SEQ ID NO: 94 - protein, 6 aa, Artificial Sequence
  Peptide conjugate
  misc_feature (1)..(1): May be protiated
  misc_feature (6)..(6): May be hydroxylated
  misc_feature (1)..(1): Xaa may be Homoserine
XCIGRL

SEQ ID NO: 95 - protein, 6 aa, Artificial Sequence
  Peptide conjugate
  misc_feature (1)..(1): May be protiated
  misc_feature (6)..(6): May be hydroxylated
  misc_feature (6)..(6): Xaa may be Norvaline
FCIGRX

SEQ ID NO: 96 - protein, 6 aa, Artificial Sequence
  Peptide conjugate
  misc_feature (1)..(1): May be protiated
  misc_feature (6)..(6): May be hydroxylated
  misc_feature (6)..(6): Xaa may be Beta-Alanine
FCIGRX

SEQ ID NO: 97 - protein, 6 aa, Artificial Sequence
  Peptide conjugate
  misc_feature (1)..(1): May be protiated
  misc_feature (6)..(6): May be hydroxylated
  misc_feature (6)..(6): Xaa may be L-tert-Leucine
FCIGRX

SEQ ID NO: 98 - protein, 6 aa, Artificial Sequence
  Peptide conjugate
  misc_feature (1)..(1): May be protiated
  misc_feature (6)..(6): May be hydroxylated
  misc_feature (6)..(6): Xaa may be L-N-methyl Alanine
FCIGRX

SEQ ID NO: 99 - protein, 6 aa, Artificial Sequence
  Peptide conjugate
  misc_feature (1)..(1): May be protiated
  misc_feature (6)..(6): May be hydroxylated
  misc_feature (6)..(6): Xaa may be L-alpha-aminobutyric acid
FCIGRX

SEQ ID NO: 100 - protein, 6 aa, Artificial Sequence
  Peptide conjugate
  misc_feature (1)..(1): May be protiated
  misc_feature (6)..(6): May be hydroxylated
  misc_feature (6)..(6): Xaa may be alpha-aminoisobutyric acid
FCIGRX

SEQ ID NO: 101 - protein, 6 aa, Artificial Sequence
  Peptide conjugate
  misc_feature (1)..(1): May be protiated
  misc_feature (6)..(6): May be hydroxylated
  misc_feature (6)..(6): Xaa may be L-beta-cyclohexyl-alanine
FCIGRX

SEQ ID NO: 102 - protein, 6 aa, Artificial Sequence
  Peptide conjugate
  misc_feature (1)..(1): May be protiated
  misc_feature (6)..(6): May be hydroxylated
  misc_feature (1)..(1): Xaa may be L-alpha-aminobutyric acid
XCIGRL

SEQ ID NO: 103 - protein, 6 aa, Artificial Sequence
  Peptide conjugate
  misc_feature (1)..(1): May be protiated
  misc_feature (6)..(6): May be hydroxylated
  misc_feature (1)..(1): Xaa may be Ornithine
XCIGRL

SEQ ID NO: 104 - protein, 6 aa, Artificial Sequence
  Peptide conjugate
  misc_feature (5)..(5): Xaa may be Citrulline
FCIGXL

SEQ ID NO: 105 - protein, 6 aa, Artificial Sequence
  Peptide conjugate
  misc_feature (1)..(1): Xaa may be L-4-nitro-phenylalanine
XCIGRL

SEQ ID NO: 106 - protein, 6 aa, Artificial Sequence
  Peptide conjugate
  misc_feature (1)..(1): Xaa may be L-4-chlorophenylalanine
XCIGRL

SEQ ID NO: 107 - protein, 6 aa, Artificial Sequence
  Peptide conjugate
  misc_feature (6)..(6): Xaa may be L-4-chlorophenylalanine
FCIGRX

SEQ ID NO: 108 - protein, 6 aa, Artificial Sequence
  Peptide conjugate
  misc_feature (6)..(6): Xaa may be L-4-nitro-phenylalanine
FCIGRX

SEQ ID NO: 109 - protein, 6 aa, Artificial Sequence
  Peptide conjugate
  misc_feature (1)..(1): Xaa may be L-1,2,3,4-tetrahydroisoquinoline-3- carboxylic acid
XCIGRL

SEQ ID NO: 110 - protein, 6 aa, Artificial Sequence
  Peptide conjugate
  misc_feature (3)..(3): Xaa may be N-methyl Isoleucine
FCXGRL

SEQ ID NO: 111 - protein, 6 aa, Artificial Sequence
  Peptide conjugate
  misc_feature (6)..(6): Xaa may be L-beta-(2-thienyl)-alanine
FCIGRX

SEQ ID NO: 112 - protein, 6 aa, Artificial Sequence
  Peptide conjugate
  misc_feature (1)..(1): Xaa may be L-beta-(2-thienyl)-alanine
XCIGRL

SEQ ID NO: 113 - protein, 6 aa, Artificial Sequence
  Peptide conjugate
  misc_feature (6)..(6): Xaa may be L-1,2,3,4-tetrahydroisoquinoline-3- carboxylic acid
FCIGRX

SEQ ID NO: 114 - protein, 6 aa, Artificial Sequence
  Peptide conjugate
  misc_feature (1)..(1): May be protiated
  misc_feature (6)..(6): May be hydroxylated
FTIGRL

SEQ ID NO: 115 - protein, 6 aa, Artificial Sequence
  Peptide conjugate
  misc_feature (1)..(1): May be protiated
  misc_feature (6)..(6): May be hydroxylated
FSIGRL

SEQ ID NO: 116 - protein, 6 aa, Artificial Sequence
  Peptide conjugate
  misc_feature (1)..(1): May be protiated
  misc_feature (6)..(6): May be hydroxylated
FMIGRL

SEQ ID NO: 117 - protein, 6 aa, Artificial Sequence
  Peptide conjugate
  misc_feature (6)..(6): May be amidated
FLIGRL

SEQ ID NO: 118 - protein, 7 aa, Artificial Sequence
  Peptide conjugate
  misc_feature (6)..(6): May be amidated
FFLIGRL

SEQ ID NO: 119 - protein, 6 aa, Artificial Sequence
  Peptide conjugate
  misc_feature (6)..(6): May be hydroxylated
FFIGRL

SEQ ID NO: 120 - protein, 6 aa, Artificial Sequence
  Peptide conjugate
  misc_feature (6)..(6): May be hydroxylated
FPIGRL

SEQ ID NO: 121 - protein, 6 aa, Artificial Sequence
  Peptide conjugate
FWIGRL

SEQ ID NO: 122 - protein, 6 aa, Artificial Sequence
  Peptide conjugate
FHIGRL

SEQ ID NO: 123 - protein, 6 aa, Artificial Sequence
  Peptide conjugate
FPIGRL

SEQ ID NO: 124 - protein, 6 aa, Artificial Sequence
  Peptide conjugate
FDIGRL

SEQ ID NO: 125 - protein, 6 aa, Artificial Sequence
  Peptide conjugate
  misc_feature (6)..(6): May be amidated
FLIGRL

SEQ ID NO: 126 - protein, 6 aa, Artificial Sequence
  Peptide conjugate
  misc_feature (6)..(6): May be amidated
FFIGRL

SEQ ID NO: 127 - protein, 6 aa, Artificial Sequence
  Peptide conjugate
FRIGRL

SEQ ID NO: 128 - protein, 6 aa, Artificial Sequence
  Peptide conjugate
FGIGRL

SEQ ID NO: 129 - protein, 6 aa, Artificial Sequence
  Peptide conjugate
FQIGRL

SEQ ID NO: 130 - protein, 6 aa, Artificial Sequence
  Peptide conjugate
FEIGRL

SEQ ID NO: 131 - protein, 6 aa, Artificial Sequence
  Peptide conjugate
FKIGRL

SEQ ID NO: 132 - protein, 6 aa, Artificial Sequence
  Peptide conjugate
FNIGRL

SEQ ID NO: 133 - protein, 6 aa, Artificial Sequence
  Peptide conjugate
FYIGRL

SEQ ID NO: 134 - protein, 6 aa, Artificial Sequence
  Peptide conjugate
FLIGRL

SEQ ID NO: 135 - protein, 6 aa, Artificial Sequence
  Peptide conjugate
FVIGRL

SEQ ID NO: 136 - protein, 6 aa, Artificial Sequence
  Peptide conjugate
FIIGRL

SEQ ID NO: 137 - protein, 6 aa, Artificial Sequence
  Peptide conjugate
  misc_feature (6)..(6): May be carboxylated
SLIGRL

SEQ ID NO: 138 - protein, 6 aa, Artificial Sequence
  Peptide conjugate
  misc_feature (6)..(6): May be amidated
LRGILF

SEQ ID NO: 139 - protein, 5 aa, Artificial Sequence
  Peptide conjugate
  misc_feature (5)..(5): May be amidated
FLIGR

SEQ ID NO: 140 - protein, 6 aa, Artificial Sequence
  Peptide conjugate
  misc_feature (1)..(1): May be protiated
  misc_feature (6)..(6): May be hydroxylated
  misc_feature (2)..(2): Xaa may be L-beta-(2-thienyl)-alanine
FXIGRL

SEQ ID NO: 141 - protein, 6 aa, Artificial Sequence
  Peptide conjugate
  misc_feature (1)..(1): May be protiated
  misc_feature (6)..(6): May be hydroxylated
  misc_feature (2)..(2): Xaa may be Homoserine
FXIGRL

SEQ ID NO: 142 - protein, 6 aa, Artificial Sequence
  Peptide conjugate
  misc_feature (1)..(1): May be protiated
  misc_feature (6)..(6): May be hydroxylated
  misc_feature (2)..(2): Xaa may be L-alpha-aminobutyric acid
FXIGRL

SEQ ID NO: 143 - protein, 6 aa, Artificial Sequence
  Peptide conjugate
  misc_feature (1)..(1): May be protiated
  misc_feature (6)..(6): May be hydroxylated
  misc_feature (2)..(2): Xaa may be L-methionine-sulfoxide
FXIGRL

SEQ ID NO: 144 - protein, 6 aa, Artificial Sequence
  Peptide conjugate
  misc_feature (1)..(1): May be protiated
  misc_feature (6)..(6): May be hydroxylated
  misc_feature (2)..(2): Xaa may be L-methionine-sulfone
FXIGRL

SEQ ID NO: 145 - protein, 6 aa, Artificial Sequence
  Peptide conjugate
  misc_feature (1)..(1): May be protiated
  misc_feature (2)..(2): Cys may be D-Cysteine
  misc_feature (6)..(6): May be hydroxylated
FCIGRL

SEQ ID NO: 146 - protein, 6 aa, Artificial Sequence
  Peptide conjugate
  misc_feature (1)..(1): May be protiated
  misc_feature (6)..(6): May be hydroxylated
  misc_feature (2)..(2): Xaa may be Norvaline
FXIGRL

SEQ ID NO: 147 - protein, 6 aa, Artificial Sequence
  Peptide conjugate
  misc_feature (1)..(1): May be protiated
  misc_feature (6)..(6): May be hydroxylated
  misc_feature (2)..(2): Xaa may be L-norleucine
FXIGRL

SEQ ID NO: 148 - protein, 6 aa, Artificial Sequence
  Peptide conjugate
  misc_feature (1)..(1): May be amidated
  misc_feature (2)..(2): Xaa may be Homoserine
FXIGRA

SEQ ID NO: 149 - protein, 6 aa, Artificial Sequence
  Peptide conjugate
  misc_feature (1)..(1): May be amidated
  misc_feature (2)..(2): Xaa may be Homoserine
FXIGRS

SEQ ID NO: 150 - protein, 6 aa, Artificial Sequence
  Peptide conjugate
  misc_feature (1)..(1): May be amidated
  misc_feature (2)..(2): Xaa may be Homoserine
FXIGRF

SEQ ID NO: 151 - protein, 6 aa, Artificial Sequence
  Peptide conjugate
  misc_feature (2)..(2): Xaa may be Homoserine
FXIGRF

SEQ ID NO: 152 - protein, 6 aa, Artificial Sequence
  Peptide conjugate
  misc_feature (2)..(2): Xaa may be L-S-benzyl-cysteine
FXIGRL

SEQ ID NO: 153 - protein, 6 aa, Artificial Sequence
  Peptide conjugate
  misc_feature (2)..(2): Xaa may be L-S-tert-butylthio-cysteine
FXIGRL

SEQ ID NO: 154 - protein, 6 aa, Artificial Sequence
  Peptide conjugate
  misc_feature (2)..(2): Xaa may be L-phenylglycine
  misc_feature (6)..(6): May be hydroxylated
FXIGRL

SEQ ID NO: 155 - protein, 6 aa, Artificial Sequence
  Peptide conjugate
  misc_feature (2)..(2): Val may be D-Val
  misc_feature (6)..(6): May be hydroxylated
FVIGRL

SEQ ID NO: 156 - protein, 6 aa, Artificial Sequence
  Peptide conjugate
  misc_feature (2)..(2): Xaa may be L-beta-cyclohexyl-alanine
  misc_feature (6)..(6): May be hydroxylated
FXIGRL

SEQ ID NO: 157 - protein, 6 aa, Artificial Sequence
  Peptide conjugate
  misc_feature (2)..(2): Xaa may be L-tert-butylglycine
  misc_feature (6)..(6): May be hydroxylated
FXIGRL

SEQ ID NO: 158 - protein, 6 aa, Artificial Sequence
  Peptide conjugate
  misc_feature (2)..(2): Xaa may be L-1,4-diaminobutyric acid
FXIGRL

SEQ ID NO: 159 - protein, 6 aa, Artificial Sequence
  Peptide conjugate
  misc_feature (2)..(2): Xaa may be L-(R,S)-3,4-cis-methanoproline
FXIGRL

SEQ ID NO: 160 - protein, 6 aa, Artificial Sequence
  Peptide conjugate
  misc_feature (2)..(2): Xaa may be L-1,3-diaminopropionic acid
FXIGRL

SEQ ID NO: 161 - protein, 6 aa, Artificial Sequence
  Peptide conjugate
  misc_feature (2)..(2): Xaa may be L-S-acetamidomethyl-penicillamine
FXIGRL

SEQ ID NO: 162 - protein, 6 aa, Artificial Sequence
  Peptide conjugate
  misc_feature (2)..(2): Xaa may be Homocysteine
FXIGRL

SEQ ID NO: 163 - protein, 6 aa, Artificial Sequence
  Peptide conjugate
  misc_feature (2)..(2): Xaa may be L-beta-(2-pyridyl)-alanine
FXIGRL

SEQ ID NO: 164 - protein, 6 aa, Artificial Sequence
  Peptide conjugate
  misc_feature (3)..(3): Ile may be D-Ile
  misc_feature (6)..(6): May be amidated
FLIGRL

SEQ ID NO: 165 - protein, 6 aa, Artificial Sequence
  Peptide conjugate
  misc_feature (2)..(2): Leu may be D-Leu
  misc_feature (6)..(6): May be amidated
FLIGRL

SEQ ID NO: 166 - protein, 6 aa, Artificial Sequence
  Peptide conjugate
  misc_feature (3)..(3): Ile may be D-Ile
  misc_feature (6)..(6): May be amidated
LRGILF

SEQ ID NO: 167 - protein, 6 aa, Artificial Sequence
  Peptide conjugate
  misc_feature (2)..(2): Xaa may be a L-alpha-aminobutyric acid dimer
FXIGRL

SEQ ID NO: 168 - protein, 6 aa, Artificial Sequence
  Peptide conjugate
  misc_feature (2)..(2): Xaa may be L-4,5-dehydro-leucine
FXIGRL

SEQ ID NO: 169 - protein, 5 aa, Artificial Sequence
  Peptide conjugate
  misc_feature (1)..(1): May be acetylated
  misc_feature (2)..(2): Xaa may be Homoserine
  misc_feature (5)..(5): May be amidated
FXIGR

SEQ ID NO: 170 - protein, 5 aa, Artificial Sequence
  Peptide conjugate
  misc_feature (2)..(2): Xaa may be Homoserine
  misc_feature (3)..(3): Ile may be D-Ile
  misc_feature (5)..(5): May be amidated
FXIGR

SEQ ID NO: 171 - protein, 6 aa, Artificial Sequence
  Peptide conjugate
  misc_feature (1)..(1): May be acetylated
  misc_feature (2)..(2): Xaa may be Homoserine
  misc_feature (6)..(6): May be amidated
FXIGRL

SEQ ID NO: 172 - protein, 6 aa, Artificial Sequence
  Peptide conjugate
  misc_feature (2)..(2): Xaa may be Homoserine
  misc_feature (3)..(3): Ile may be D-Ile
FXIGRL

SEQ ID NO: 173 - protein, 6 aa, Artificial Sequence
  Peptide conjugate
  misc_feature (1)..(1): Xaa may be L-4-cyano-phenylalanine
XFIGRL

SEQ ID NO: 174 - protein, 6 aa, Artificial Sequence
  Peptide conjugate
  misc_feature (1)..(1): Xaa may be L-3-methyl-phenylalanine
XFIGRL

SEQ ID NO: 175 - protein, 6 aa, Artificial Sequence
  Peptide conjugate
  misc_feature (2)..(2): Xaa may be L-beta-cyclopropyl-alanine
FXIGRL

SEQ ID NO: 176 - protein, 5 aa, Artificial Sequence
  Peptide conjugate
  misc_feature (2)..(2): Xaa may be L-propargylglycine
  misc_feature (5)..(5): May be amidated
FXIGR

SEQ ID NO: 177 - protein, 5 aa, Artificial Sequence
  Peptide conjugate
  misc_feature (2)..(2): Xaa may be L-beta-(2-furyl)-alanine
  misc_feature (5)..(5): May be amidated
FXIGR

SEQ ID NO: 178 - protein, 5 aa, Artificial Sequence
  Peptide conjugate
  misc_feature (2)..(2): Xaa may be 1,2,3,4-tetrahydroharmane-3- carboxylic acid
  misc_feature (5)..(5): May be amidated
FXIGR

SEQ ID NO: 179 - protein, 6 aa, Artificial Sequence
  Peptide conjugate
  misc_feature (2)..(2): Xaa may be L-beta-styryl-alanine
  misc_feature (6)..(6): May be amidated
FXIGRL

SEQ ID NO: 180 - protein, 6 aa, Artificial Sequence
  Peptide conjugate
  misc_feature (2)..(2): Xaa may be Homocitrulline
  misc_feature (6)..(6): May be amidated
FXIGRL

SEQ ID NO: 181 - protein, 6 aa, Artificial Sequence
  Peptide conjugate
  misc_feature (2)..(2): Xaa may be 1,2,3,4-tetrahydroharmane-3- carboxylic acid
  misc_feature (6)..(6): May be amidated
FXIGRL

SEQ ID NO: 182 - protein, 6 aa, Artificial Sequence
  Peptide conjugate
  misc_feature (2)..(2): Xaa may be L-beta-(2-furyl)-alanine
  misc_feature (6)..(6): May be amidated
FXIGRL

SEQ ID NO: 183 - protein, 6 aa, Artificial Sequence
  Peptide conjugate
  misc_feature (2)..(2): Xaa may be L-allylglycine
  misc_feature (6)..(6): May be hydroxylated
FXIGRL

SEQ ID NO: 184 - protein, 5 aa, Artificial Sequence
  Peptide conjugate
  misc_feature (2)..(2): Xaa may be L-allylglycine
  misc_feature (5)..(5): May be amidated
FXIGR

SEQ ID NO: 185 - protein, 5 aa, Artificial Sequence
  Peptide conjugate
  misc_feature (2)..(2): Xaa may be D-allylglycine
  misc_feature (5)..(5): May be amidated
FXIGR

SEQ ID NO: 186 - protein, 6 aa, Artificial Sequence
  Peptide conjugate
  misc_feature (2)..(2): Xaa may be L-allylglycine
  misc_feature (6)..(6): May be amidated
MXIGRL

SEQ ID NO: 187 - protein, 6 aa, Artificial Sequence
  Peptide conjugate
  misc_feature (2)..(2): Xaa may be L-allylglycine
  misc_feature (6)..(6): May be amidated
QXIGRL

SEQ ID NO: 188 - protein, 6 aa, Artificial Sequence
  Peptide conjugate
  misc_feature (2)..(2): Xaa may be L-allylglycine
  misc_feature (6)..(6): May be amidated
LXIGRL

SEQ ID NO: 189 - protein, 6 aa, Artificial Sequence
  Peptide conjugate
  misc_feature (2)..(2): Xaa may be L-allylglycine
  misc_feature (6)..(6): May be amidated
SXIGRL

SEQ ID NO: 190 - protein, 6 aa, Artificial Sequence
  Peptide conjugate
  misc_feature (2)..(2): Xaa may be L-allylglycine
  misc_feature (6)..(6): May be amidated
TXIGRL

SEQ ID NO: 191 - protein, 6 aa, Artificial Sequence
  Peptide conjugate
  misc_feature (2)..(2): Xaa may be L-allylglycine
  misc_feature (6)..(6): May be amidated
EXIGRL

SEQ ID NO: 192 - protein, 6 aa, Artificial Sequence
  Peptide conjugate
  misc_feature (2)..(2): Xaa may be L-allylglycine
  misc_feature (6)..(6): May be amidated
VXIGRL

SEQ ID NO: 193 - protein, 6 aa, Artificial Sequence
  Peptide conjugate
  misc_feature (2)..(2): Xaa may be L-allylglycine
  misc_feature (6)..(6): May be amidated
YXIGRL

SEQ ID NO: 194 - protein, 6 aa, Artificial Sequence
  Peptide conjugate
  misc_feature (2)..(2): Xaa may be L-allylglycine
  misc_feature (6)..(6): May be amidated
GXIGRL

SEQ ID NO: 195 - protein, 6 aa, Artificial Sequence
  Peptide conjugate
  misc_feature (2)..(2): Xaa may be L-allylglycine
  misc_feature (6)..(6): May be amidated
DXIGRL

SEQ ID NO: 196 - protein, 6 aa, Artificial Sequence
  Peptide conjugate
  misc_feature (2)..(2): Xaa may be L-allylglycine
  misc_feature (6)..(6): May be amidated
WXIGRL

SEQ ID NO: 197 - protein, 6 aa, Artificial Sequence
  Peptide conjugate
  misc_feature (2)..(2): Xaa may be L-allylglycine
  misc_feature (6)..(6): May be amidated
KXIGRL

SEQ ID NO: 198 - protein, 6 aa, Artificial Sequence
  Peptide conjugate
  misc_feature (2)..(2): Xaa may be L-allylglycine
  misc_feature (6)..(6): May be amidated
AXIGRL

SEQ ID NO: 199 - protein, 6 aa, Artificial Sequence
  Peptide conjugate
  misc_feature (2)..(2): Xaa may be L-allylglycine
  misc_feature (6)..(6): May be amidated
HXIGRL

SEQ ID NO: 200 - protein, 6 aa, Artificial Sequence
  Peptide conjugate
  misc_feature (2)..(2): Xaa may be L-allylglycine
  misc_feature (6)..(6): May be amidated
PXIGRL

SEQ ID NO: 201 - protein, 6 aa, Artificial Sequence
  Peptide conjugate
  misc_feature (2)..(2): Xaa may be L-allylglycine
  misc_feature (6)..(6): May be amidated
RXIGRL

SEQ ID NO: 202 - protein, 6 aa, Artificial Sequence
  Peptide conjugate
  misc_feature (2)..(2): Xaa may be L-allylglycine
  misc_feature (6)..(6): May be amidated
IXIGRL

SEQ ID NO: 203 - protein, 6 aa, Artificial Sequence
  Peptide conjugate
  misc_feature (2)..(2): Xaa may be L-allylglycine
  misc_feature (6)..(6): May be amidated
FXPGRL

SEQ ID NO: 204 - protein, 6 aa, Artificial Sequence
  Peptide conjugate
  misc_feature (2)..(2): Xaa may be L-allylglycine
  misc_feature (6)..(6): May be amidated
FXFGRL

SEQ ID NO: 205 - protein, 6 aa, Artificial Sequence
  Peptide conjugate
  misc_feature (2)..(2): Xaa may be L-allylglycine
  misc_feature (6)..(6): May be amidated
FXTGRL

SEQ ID NO: 206 - protein, 6 aa, Artificial Sequence
  Peptide conjugate
  misc_feature (2)..(2): Xaa may be L-allylglycine
  misc_feature (6)..(6): May be amidated
FXLGRL

SEQ ID NO: 207 - protein, 6 aa, Artificial Sequence
  Peptide conjugate
  misc_feature (2)..(2): Xaa may be L-allylglycine
  misc_feature (6)..(6): May be amidated
FXSGRL

SEQ ID NO: 208 - protein, 6 aa, Artificial Sequence
  Peptide conjugate
  misc_feature (2)..(2): Xaa may be L-allylglycine
  misc_feature (6)..(6): May be amidated
FXFGRL

SEQ ID NO: 209 - protein, 6 aa, Artificial Sequence
  Peptide conjugate
  misc_feature (2)..(2): Xaa may be L-allylglycine
  misc_feature (6)..(6): May be amidated
FXVGRL

SEQ ID NO: 210 - protein, 6 aa, Artificial Sequence
  Peptide conjugate
  misc_feature (2)..(2): Xaa may be L-allylglycine
  misc_feature (6)..(6): May be amidated
FXGGRL

SEQ ID NO: 211 - protein, 6 aa, Artificial Sequence
  Peptide conjugate
  misc_feature (2)..(2): Xaa may be L-allylglycine
  misc_feature (6)..(6): May be amidated
FXAGRL

SEQ ID NO: 212 - protein, 6 aa, Artificial Sequence
  Peptide conjugate
  misc_feature (2)..(2): Xaa may be L-allylglycine
  misc_feature (6)..(6): May be amidated
FXHGRL

SEQ ID NO: 213 - protein, 6 aa, Artificial Sequence
  Peptide conjugate
  misc_feature (2)..(2): Xaa may be L-allylglycine
  misc_feature (6)..(6): May be amidated
FXDGRL

SEQ ID NO: 214 - protein, 6 aa, Artificial Sequence
  Peptide conjugate
  misc_feature (2)..(2): Xaa may be L-allylglycine
  misc_feature (6)..(6): May be amidated
FXEGRL

SEQ ID NO: 215 - protein, 6 aa, Artificial Sequence
  Peptide conjugate
  misc_feature (2)..(2): Xaa may be L-allylglycine
  misc_feature (6)..(6): May be amidated
FXQGRL

SEQ ID NO: 216 - protein, 6 aa, Artificial Sequence
  Peptide conjugate
  misc_feature (2)..(2): Xaa may be L-allylglycine
  misc_feature (6)..(6): May be amidated
FXRGRL

SEQ ID NO: 217 - protein, 6 aa, Artificial Sequence
  Peptide conjugate
  misc_feature (2)..(2): Xaa may be L-allylglycine
  misc_feature (6)..(6): May be amidated
FXKGRL

SEQ ID NO: 218 - protein, 6 aa, Artificial Sequence
  Peptide conjugate
  misc_feature (2)..(2): Xaa may be L-allylglycine
  misc_feature (6)..(6): May be amidated
FXNGRL

SEQ ID NO: 219 - protein, 6 aa, Artificial Sequence
  Peptide conjugate
  misc_feature (2)..(2): Xaa may be L-allylglycine
  misc_feature (6)..(6): May be amidated
FXYGRL

SEQ ID NO: 220 - protein, 6 aa, Artificial Sequence
  Peptide conjugate
  misc_feature (2)..(2): Xaa may be L-allylglycine
  misc_feature (6)..(6): May be amidated
FXITRL

SEQ ID NO: 221 - protein, 6 aa, Artificial Sequence
  Peptide conjugate
  misc_feature (2)..(2): Xaa may be L-allylglycine
  misc_feature (6)..(6): May be amidated
FXILRL

SEQ ID NO: 222 - protein, 6 aa, Artificial Sequence
  Peptide conjugate
  misc_feature (2)..(2): Xaa may be L-allylglycine
  misc_feature (6)..(6): May be amidated
FXIIRL

SEQ ID NO: 223 - protein, 6 aa, Artificial Sequence
  Peptide conjugate
  misc_feature (2)..(2): Xaa may be L-allylglycine
  misc_feature (6)..(6): May be amidated
FXIARL

SEQ ID NO: 224 - protein, 6 aa, Artificial Sequence
  Peptide conjugate
  misc_feature (2)..(2): Xaa may be L-allylglycine
  misc_feature (6)..(6): May be amidated
FXIPRL

SEQ ID NO: 225 - protein, 6 aa, Artificial Sequence
  Peptide conjugate
  misc_feature (2)..(2): Xaa may be L-allylglycine
  misc_feature (6)..(6): May be amidated
FXIGRL

SEQ ID NO: 226 - protein, 6 aa, Artificial Sequence
  Peptide conjugate
  misc_feature (2)..(2): Xaa may be L-allylglycine
  misc_feature (6)..(6): May be amidated
FXIHRL

SEQ ID NO: 227 - protein, 6 aa, Artificial Sequence
  Peptide conjugate
  misc_feature (2)..(2): Xaa may be L-allylglycine
  misc_feature (6)..(6): May be amidated
FXIDRL

SEQ ID NO: 228 - protein, 6 aa, Artificial Sequence
  Peptide conjugate
  misc_feature (2)..(2): Xaa may be L-allylglycine
  misc_feature (6)..(6): May be amidated
FXIERL

SEQ ID NO: 229 - protein, 6 aa, Artificial Sequence
  Peptide conjugate
  misc_feature (2)..(2): Xaa may be L-allylglycine
  misc_feature (6)..(6): May be amidated
FXIQRL

SEQ ID NO: 230 - protein, 6 aa, Artificial Sequence
  Peptide conjugate
  misc_feature (2)..(2): Xaa may be L-allylglycine
  misc_feature (6)..(6): May be amidated
FXIFRL

SEQ ID NO: 231 - protein, 6 aa, Artificial Sequence
  Peptide conjugate
  misc_feature (2)..(2): Xaa may be L-allylglycine
  misc_feature (6)..(6): May be amidated
FXIRRL

SEQ ID NO: 232 - protein, 6 aa, Artificial Sequence
  Peptide conjugate
  misc_feature (2)..(2): Xaa may be L-allylglycine
  misc_feature (6)..(6): May be amidated
FXIKRL

SEQ ID NO: 233 - protein, 6 aa, Artificial Sequence
  Peptide conjugate
  misc_feature (2)..(2): Xaa may be L-allylglycine
  misc_feature (6)..(6): May be amidated
FXINRL

SEQ ID NO: 234 - protein, 6 aa, Artificial Sequence
  Peptide conjugate
  misc_feature (2)..(2): Xaa may be L-allylglycine
  misc_feature (6)..(6): May be amidated
FXISRL

SEQ ID NO: 235 - protein, 6 aa, Artificial Sequence
  Peptide conjugate
  misc_feature (2)..(2): Xaa may be L-allylglycine
  misc_feature (6)..(6): May be amidated
FXIVRL

SEQ ID NO: 236 - protein, 6 aa, Artificial Sequence
  Peptide conjugate
  misc_feature (2)..(2): Xaa may be L-allylglycine
  misc_feature (6)..(6): May be amidated
FXIGHL

SEQ ID NO: 237 - protein, 6 aa, Artificial Sequence
  Peptide conjugate
  misc_feature (2)..(2): Xaa may be L-allylglycine
  misc_feature (6)..(6): May be amidated
FXIGDL

SEQ ID NO: 238 - protein, 6 aa, Artificial Sequence
  Peptide conjugate
  misc_feature (2)..(2): Xaa may be L-allylglycine
  misc_feature (6)..(6): May be amidated
FXIGEL

SEQ ID NO: 239 - protein, 6 aa, Artificial Sequence
  Peptide conjugate
  misc_feature (2)..(2): Xaa may be L-allylglycine
  misc_feature (6)..(6): May be amidated
FXIGQL

SEQ ID NO: 240 - protein, 6 aa, Artificial Sequence
  Peptide conjugate
  misc_feature (2)..(2): Xaa may be L-allylglycine
  misc_feature (6)..(6): May be amidated
FXIGGL

SEQ ID NO: 241 - protein, 6 aa, Artificial Sequence
  Peptide conjugate
  misc_feature (2)..(2): Xaa may be L-allylglycine
  misc_feature (6)..(6): May be amidated
FXIGAL

SEQ ID NO: 242 - protein, 6 aa, Artificial Sequence
  Peptide conjugate
  misc_feature (2)..(2): Xaa may be L-allylglycine
  misc_feature (6)..(6): May be amidated
FXIGFL

SEQ ID NO: 243 - protein, 6 aa, Artificial Sequence
  Peptide conjugate
  misc_feature (2)..(2): Xaa may be L-allylglycine
  misc_feature (6)..(6): May be amidated
FXIGKL

SEQ ID NO: 244 - protein, 6 aa, Artificial Sequence
  Peptide conjugate
  misc_feature (2)..(2): Xaa may be L-allylglycine
  misc_feature (6)..(6): May be amidated
FXIGLL

SEQ ID NO: 245 - protein, 6 aa, Artificial Sequence
  Peptide conjugate
  misc_feature (2)..(2): Xaa may be L-allylglycine
  misc_feature (6)..(6): May be amidated
FXIGML

SEQ ID NO: 246 - protein, 6 aa, Artificial Sequence
  Peptide conjugate
  misc_feature (2)..(2): Xaa may be L-allylglycine
  misc_feature (6)..(6): May be amidated
FXIGNL

SEQ ID NO: 247 - protein, 6 aa, Artificial Sequence
  Peptide conjugate
  misc_feature (2)..(2): Xaa may be L-allylglycine
  misc_feature (6)..(6): May be amidated
FXIGSL

SEQ ID NO: 248 - protein, 6 aa, Artificial Sequence
  Peptide conjugate
  misc_feature (2)..(2): Xaa may be L-allylglycine
  misc_feature (6)..(6): May be amidated
FXIGYL

SEQ ID NO: 249 - protein, 6 aa, Artificial Sequence
  Peptide conjugate
  misc_feature (2)..(2): Xaa may be L-allylglycine
  misc_feature (6)..(6): May be amidated
FXIGTL

SEQ ID NO: 250 - protein, 6 aa, Artificial Sequence
  Peptide conjugate
  misc_feature (2)..(2): Xaa may be L-allylglycine
  misc_feature (6)..(6): May be amidated
FXIGIL

SEQ ID NO: 251 - protein, 6 aa, Artificial Sequence
  Peptide conjugate
  misc_feature (2)..(2): Xaa may be L-allylglycine
  misc_feature (6)..(6): May be amidated
FXIGWL

SEQ ID NO: 252 - protein, 6 aa, Artificial Sequence
  Peptide conjugate
  misc_feature (2)..(2): Xaa may be L-allylglycine
  misc_feature (6)..(6): May be amidated
FXIGPL

SEQ ID NO: 253 - protein, 6 aa, Artificial Sequence
  Peptide conjugate
  misc_feature (2)..(2): Xaa may be L-allylglycine
  misc_feature (6)..(6): May be amidated
FXIGVL

SEQ ID NO: 254 - protein, 6 aa, Artificial Sequence
  Peptide conjugate
  misc_feature (2)..(2): Xaa may be L-allylglycine
  misc_feature (6)..(6): May be amidated
FXIGRH

SEQ ID NO: 255 - protein, 6 aa, Artificial Sequence
  Peptide conjugate
  misc_feature (2)..(2): Xaa may be L-allylglycine
  misc_feature (6)..(6): May be amidated
FXIGRD

SEQ ID NO: 256 - protein, 6 aa, Artificial Sequence
  Peptide conjugate
  misc_feature (2)..(2): Xaa may be L-allylglycine
  misc_feature (6)..(6): May be amidated
FXIGRR

SEQ ID NO: 257 - protein, 6 aa, Artificial Sequence
  Peptide conjugate
  misc_feature (2)..(2): Xaa may be L-allylglycine
  misc_feature (6)..(6): May be amidated
FXIGRF

SEQ ID NO: 258 - protein, 6 aa, Artificial Sequence
  Peptide conjugate
  misc_feature (2)..(2): Xaa may be L-allylglycine
  misc_feature (6)..(6): May be amidated
FXIGRA

SEQ ID NO: 259 - protein, 6 aa, Artificial Sequence
  Peptide conjugate
  misc_feature (2)..(2): Xaa may be L-allylglycine
  misc_feature (6)..(6): May be amidated
FXIGRG

SEQ ID NO: 260 - protein, 6 aa, Artificial Sequence
  Peptide conjugate
  misc_feature (2)..(2): Xaa may be L-allylglycine
  misc_feature (6)..(6): May be amidated
FXIGRQ

SEQ ID NO: 261 - protein, 6 aa, Artificial Sequence
  Peptide conjugate
  misc_feature (2)..(2): Xaa may be L-allylglycine
  misc_feature (6)..(6): May be amidated
FXIGRE

SEQ ID NO: 262 - protein, 6 aa, Artificial Sequence
  Peptide conjugate
  misc_feature (2)..(2): Xaa may be L-allylglycine
  misc_feature (6)..(6): May be amidated
FXIGRT

SEQ ID NO: 263 - protein, 6 aa, Artificial Sequence
  Peptide conjugate
  misc_feature (2)..(2): Xaa may be L-allylglycine
  misc_feature (6)..(6): May be amidated
FXIGRY

SEQ ID NO: 264 - protein, 6 aa, Artificial Sequence
  Peptide conjugate
  misc_feature (2)..(2): Xaa may be L-allylglycine
  misc_feature (6)..(6): May be amidated
FXIGRS

SEQ ID NO: 265 - protein, 6 aa, Artificial Sequence
  Peptide conjugate
  misc_feature (2)..(2): Xaa may be L-allylglycine
  misc_feature (6)..(6): May be amidated
FXIGRN

SEQ ID NO: 266 - protein, 6 aa, Artificial Sequence
  Peptide conjugate
  misc_feature (2)..(2): Xaa may be L-allylglycine
  misc_feature (6)..(6): May be amidated
FXIGRM

SEQ ID NO: 267-276 - skipped (10 entries)

SEQ ID NO: 277 - protein, 6 aa, Artificial Sequence
  Peptide conjugate
  misc_feature (2)..(2): Xaa may be L-allylglycine
  misc_feature (6)..(6): May be amidated
FXIGRK

SEQ ID NO: 278-287 - skipped (10 entries)

SEQ ID NO: 288 - protein, 6 aa, Artificial Sequence
  Peptide conjugate
  misc_feature (2)..(2): Xaa may be L-allylglycine
  misc_feature (6)..(6): May be amidated
FXIGRI

SEQ ID NO: 289 - protein, 6 aa, Artificial Sequence
  Peptide conjugate
  misc_feature (2)..(2): Xaa may be L-allylglycine
  misc_feature (6)..(6): May be amidated
FXIGRW

SEQ ID NO: 290 - protein, 6 aa, Artificial Sequence
  Peptide conjugate
  misc_feature (2)..(2): Xaa may be L-allylglycine
  misc_feature (6)..(6): May be amidated
FXIGRP

SEQ ID NO: 291 - protein, 6 aa, Artificial Sequence
  Peptide conjugate
  misc_feature (2)..(2): Xaa may be L-allylglycine
  misc_feature (6)..(6): May be amidated
FXIGRV

(19) United States
(12) Patent Application Publication
(10) Pub. No.: US 2012/0027720 A1
(43) Pub. Date: Feb. 2, 2012

(54) PEPTIDE CONJUGATES

(76) Inventors: Amir TAMIZ, Silver Spring, MD (US); Sefik Alkan, Baltimore, MD (US)

(21) Appl. No.: 13/168,734

(22) Filed: Jun. 24, 2011

Related U.S. Application Data
(63) Continuation of application No. 12/991,397, now abandoned.
(60) Provisional application No. 61/050,395, filed on May 5, 2008.

(51) Int. Cl. A61K 38/08; A61K 38/07; A61P 3/10; A61K 39/385; A61K 39/395; A61K 38/20; A61K 38/18; C07K 17/00; A61P 35/00

(52) U.S. Cl. 424/85.2; 530/330; 530/329; 514/21.9; 514/21.8; 514/6.9; 514/19.3; 530/391.7; 530/322; 530/351; 424/178.1; 514/7.6; 514/21.7; 424/193.1

(57) ABSTRACT

The present invention provides novel peptide conjugates. Peptide conjugates of the invention can be used as therapeutic agents. Peptide conjugates invention may also be used to deliver one or more additional active agents. The present invention also provides methods for the treatment of disease by administering to a subject suffering from the disease a composition comprising a peptide conjugate of the invention, optionally.

### PRIORITY

This application claims priority to U.S. Provisional Application No. 61/050,395 filed May 5, 2008, which is hereby incorporated by reference.

### FIELD OF THE INVENTION

The present invention provides novel peptide conjugates, their use as therapeutic agents and their use in materials and methods to facilitate the delivery of therapeutic agents. In some embodiments, novel conjugates are used in compositions to facilitate the uptake of therapeutic agents across biological barriers comprising tight junctions. In some embodiments, novel peptide conjugates are used in compositions to facilitate the uptake of therapeutic agents across biological membranes. In particular embodiments, novel peptide conjugates are covalently linked to therapeutic agents by non-cleavable linkers. In particular embodiments, novel peptide conjugates are covalently linked to therapeutic agents by cleavable linkers. In some embodiments, novel peptide conjugates are used in compositions to modulate an immune response in a subject. In some embodiments, novel peptide conjugates are used in compositions to raise an immune response against an antigen.

### BACKGROUND OF THE INVENTION

Methods and compositions for increasing absorption and bioavailability of bioactive molecules have gained significant attention for enhancing therapeutic levels of small molecule drugs (anionic, cationic or neutrally charged) or nucleic acids, peptides and proteins to treat various mammalian diseases. In addition to the delivery of drugs, gene mediated therapy holds the promise of treating or correcting underlying genetic deficiencies or defects without long term continuous treatment.

### Small Molecule Drugs

The low bioavailability (BA) of efficacious pharmacotherapeutic drugs continues to be a major obstacle in drug development and in many instances may be the deciding factor on whether or not a potent agent is developed. Many therapeutic agents experience low BA after oral administration due to poor absorption or susceptibility to first pass metabolism. A means of enhancing the gastrointestinal absorption of such drugs would significantly extend their therapeutic usefulness while decreasing the dose required to produce efficacy.

Absorption enhancers, including surfactants, fatty acids, and chitosan derivatives, have been used to modify bioavailability by either disruption of the cell membrane or modulation of the tight junctions (TJ). In general, the optimal absorption enhancer should possess the following qualities: its effect should be reversible, it should provide a rapid permeation enhancing effect on the intestinal cellular membrane, it should be non-cytotoxic at the effective concentration level without deleterious and/or irreversible effects on the cellular membrane or cytoskeleton of the TJ. Zonula Occludens Toxin (Zot), a 44.8 kDa protein (399 amino acids; AA) located in the cell envelope of the bacterial strain Vibrio cholerae, is capable of reversibly opening the TJ between cells and increasing the paracellular transport of many drugs in a non-toxic manner. Intensive investigation of the biological activity of Zot as an absorption enhancer was triggered by reports of effective oral administration of insulin with Zot in diabetic rats. Recently, a smaller 12 kDa fragment (AA 265-399) of Zot, referred to as delta G (ΔG), was introduced as the biologically active fragment of Zot. Amino acid analysis of Zot active fragment combined with site-directed mutagenesis experiments, confirmed the presence of an octapeptide receptor-binding domain toward the amino terminus of the processed Zot.

Applicants disclose novel peptide conjugates that enhance tight junction permeability, and methods of increasing bioavailability of pharmacotherapeutic drugs. The novel peptide conjugates facilitate transport of pharmacotherapeutic drugs across biological barriers whose permeability is regulated by tight junctions and thereby allows for increased bioavailability of such drugs. The peptide portions of the novel peptide conjugates of the present invention are advantageous in that they are non-toxic, their effects are reversible, they are devoid of endotoxin contamination, readily synthesized and inexpensive to produce and purify.

### Nucleic Acids

Gene therapy has garnered considerable attention as a method to treat various human diseases by the enhancement of protein production. These include gene replacement or gene augmentation. However, the delivery of genetic material into multicellular organisms has proven difficult. A large number of techniques have been developed to accomplish in vivo transformation of cells including direct injection of nucleic acid or a particle decorated with nucleic acid directly into cells, recombinant viruses, liposomes and receptor mediated endocytosis. However, these methodologies have been of limited usefulness in the treatment of disease in human patients.

Several other kinds of potential nucleic acid therapeutics have been explored, including RNA inhibitors such as antisense, ribozymes (catalytic RNAs), and artificial ligand inhibitors (“aptamers”). These therapeutics are designed to silence gene expression, and thus to alleviate the effects of undesirable genes, be they endogenous to an organism or exogenous, such as bacterial or viral in origin. However, expression of these therapeutics intracellularly has proved quite difficult due to several factors. These difficulties include, for example, the considerations of finding their targets, folding into the effective configuration, and possibly interacting with the appropriate proteins while avoiding interactions with inappropriate proteins. There have been isolated promising results (see, for example, Bertrand, E et al. (1997) RNA 3: 75-88; Good, P D et al. (1997) Gene Therapy 4: 45-54), but no therapeutics have yet resulted.

Recently the field of reverse genetic analysis, or gene silencing, has been revolutionized by the discovery of potent, sequence specific inactivation of gene function, which can be induced by double-stranded RNA (dsRNA). In contrast to the limited effectiveness of inhibiting gene expression with antisense, ribozymes, and aptamers, “RNA interference” (RNAi) works quite well to suppress expression of a gene's RNA in lower eukaryotes. RNAi is the use of double-stranded RNA to silence the expression of specific mRNAs, where it is believed that the targeted RNA is degraded, although this has not yet been confirmed. The active agent in RNAi is a long double-stranded (antiparallel duplex) RNA, with one of the strands corresponding or complementary to the RNA which is to be inhibited.

RNAi was originally shown to work in human cells if the RNA strands were provided as pre-sized duplexes of about 19 nucleotide pairs, and RNAi worked particularly well with small unpaired 3′ extensions on the end of each strand (Elbashir et al. (2001) Nature 411: 494-498). Later reports demonstrated that “short interfering RNA” (siRNA, also referred to as small interfering RNA) that are too short to elicit sequence-nonspecific responses like apoptosis, can efficiently initiate RNAi. This discovery has allowed almost unlimited experiments to knock out any known gene in mammalian cells, and the reports indiacte that siRNA appears to work quite well in most instances, far better and more consistently than do ribozymes, antisense or other nucleic acid agents. However, a major limitation to the use of siRNA in mammalian cells is the method of its delivery.

Currently, the synthesis of the siRNA is expensive. Moreover, inducing cells to take up exogenous nucleic acids is a short-term treatment and is very difficult to achieve in some cultured cell types. This methodology does not permit long-term expression of the siRNA in cells or use of siRNA in tissues, organs, and whole organisms. It had also not been demonstrated that siRNA could effectively be expressed from recombinant DNA constructs to suppress expression of a target gene. Thus, what is needed are methods to express and deliver siRNA intracellularly in mammalian cells, and indeed in other cells as well.

Thus, there exists a need for improved nucleic acid, peptide, protein and small molecule delivery agents amenable to efficient transfection of target cells and capable of inducing systemic and/or local transfection and therapeutic delivery. Applicants disclose novel peptide conjugates that enhance the intracellular delivery of nucleic acids. The novel peptide conjugates facilitate transport of nucleic acids across biological membranes and thereby allows for increased intracellular availability of such compounds. The novel peptide conjugates of the present invention are advantageous in that they are non-toxic, their effects are reversible, they are devoid of endotoxin contamination, readily synthesized and inexpensive to produce and purify.

### Vaccines

Vaccines have proven to be successful, highly acceptable methods for the prevention of infectious diseases. They are cost effective, and do not induce antibiotic resistance to the target pathogen or affect normal flora present in the host. In many cases, such as when inducing anti-viral immunity, vaccines can prevent a disease for which there are no viable curative or ameliorative treatments available.

As is well known in the art, vaccines function by triggering the immune system to mount a response to an immunogenic agent, or antigen (antigenic agent), typically an infectious organism or a portion thereof that is introduced into the body in a non-infectious or non-pathogenic form. Once the immune system has been “primed” or sensitized to the organism, later exposure of the immune system to this organism as an infectious pathogen results in a rapid and robust immune response that destroys the pathogen before it can multiply and infect enough cells in the host organism to cause disease symptoms. The agent or antigen used to induce the immune system can be the entire organism in a less infectious state, known as an attenuated organism, or in some cases, components of the organism such as carbohydrates, proteins or peptides representing various structural components of the organism.

In many cases, it is necessary to enhance the immune response to the antigens present in a vaccine in order to stimulate the immune system to a sufficient extent to make a vaccine effective, i.e., to confer immunity. Many protein and most peptide and carbohydrate antigens, administered alone, do not elicit a sufficient antibody response to confer immunity. Such antigens need to be presented to the immune system in such a way that they will be recognized as foreign and will elicit an immune response. To this end, adjuvants have been devised which stimulate the immune response.

The best known adjuvant, Freund's complete adjuvant, consists of a mixture of mycobacteria in an oil/water emulsion. Freund's adjuvant works in two ways: first, by enhancing cell and humoral-mediated immunity, and second, by blocking rapid dispersal of the antigen challenge (the “depot effect”). However, due to frequent toxic physiological and immunological reactions to this material, Freund's adjuvant cannot be used in humans. Another molecule that has been shown to have immunostimulatory or adjuvant activity is endotoxin, also known as lipopolysaccharide (LPS). LPS stimulates the immune system by triggering an “innate” immune response—a response that has evolved to enable an organism to recognize endotoxin (and the invading bacteria of which it is a component) without the need for the organism to have been previously exposed. While LPS is too toxic to be a viable adjuvant, molecules that are structurally related to endotoxin, such as monophosphoryl lipid A (“MPL”) are being tested as adjuvants in clinical trials. Currently, however, the only FDA-approved adjuvant for use in humans is aluminum salts (Alum) which are used to “depot” antigens by precipitation of the antigens. Alum also stimulates the immune response to antigens.

Thus, there is a recognized need in the art for compounds which can be co-administered with antigens in order to stimulate the immune system to generate a more robust antibody response to the antigen than would be seen if the antigen were injected alone or with Alum. Further, because development of mucosal vaccines requires the use of specific adjuvants, adjuvants that work for systemic immunization such as Alum are generally not effective for mucosal immunization. Despite intensive research on adjuvants for mucosal vaccines in the last decade, no adjuvants have been registered for human use so far. The main issues in adjuvant research are efficacy and toxicity, and candidate mucosal adjuvants do not completely satisfy the criteria of high efficacy and absence of toxicity. Furthermore, most of the proposed mucosal adjuvants are complex molecules whose mechanism of action is poorly understood. Applicants provide herein non-toxic alternative peptide conjgate adjuvants for inducing immune responses to an antigen.

Zonula Occludens Toxin (ZOT) from Vibrio cholerae was identified as an adjuvant for mucosal vaccination (Infect. Immun. 1999, 67:1287; Infect. Immun. 2003, 71:1897). Intranasal administration of ZOT with a soluble antigen in mice stimulated systemic humoral and cell-mediated responses as well as mucosal responses specific for the antigen Ovalbumin (Infect. Immun. 2003, 71:1897). ZOT is a protein of 44.8 kDa that binds a receptor on epithelial cells and modulates tight junctions, inducing the increase of mucosal barrier permeability. The effect of ZOT on tight junctions is reversible and does not cause tissue damage (J. Clin. Invest. 1995, 96:710). The receptor for ZOT on epithelial cells has been partially characterized and recently a mammalian protein with homology to ZOT has been identified and named Zonulin. Interestingly, this protein has been shown to be an endogenous regulator of tight junctions that is released by epithelial cells and binds to the same receptor used by ZOT (Ann. NY. Acad. Sci. 2000, 915:214). The mechanism of ZOT as an adjuvant may involve binding to its receptor on the nasal mucosa, modulation of tight junctions and antigen passage in the submucosa, with subsequent exposure to cells of the immune system.

The development of mucosal vaccines for the prevention of infectious diseases is highly desirable. Mucosal vaccination has several advantages over parenteral vaccination. Mucosal immunization induces an immune response at the site of infection (locally). Furthermore, because of the intrinsic properties of the mucosal immune system, the immunization at one mucosal site can induce specific responses at distant sites (regionally). Such flexibility is important to address cultural and religious barriers to vaccination because protective immunity (for instance against sexually-transmitted diseases) may then be induced in segregated mucosal sites in a practical way. In addition to local responses against mucosally-acquired pathogens, mucosal vaccines induce systemic immunity, including humoral and cell-mediated responses. Thus, mucosal vaccination could be exploited for combating infections acquired through other routes (i.e., blood or skin). Finally, the administration of mucosal vaccines does not require the use of needles, which could increase vaccine compliance and negate concerns with blood transmissible infections. For all the above reasons mucosal vaccines may be used also to combat cancer, either with preventive or therapeutic vaccination. These vaccines may be both against cancers caused by infectious agents (such as Helicobacter pylori, Papilloma Virus, Herpes Virus) and cancers of different etiology (such as melanoma, colon cancer and others).

Interestingly, most human pathogens are acquired through the mucosal route, however, few mucosal vaccines are presently used. Of those currently used, the vaccine is based on a living attenuated microorganism. Further, purified antigens are not able to stimulate/induce an immune response per se when delivered at mucosal surfaces. Therefore, such vaccines require the use of specific adjuvants. Unfortunately, development of mucosal vaccines has been so far hampered by the lack of safe and effective adjuvants as described above. An effective mucosal adjuvant allows antigen (Ag) passage through a mucosal barrier and facilitates the induction of an Ag-specific immune response.

Applicants disclose novel peptide conjugates that enhance tight junction permeability, and methods of mucosal delivery of an antigen in such peptide conjugates to induce systemic and/or mucosal responses specific for the antigen. The novel peptide conjugates facilitate delivery of the antigen through the mucosa and induce systemic and mucosal responses to the antigen. The novel peptide conjugates of the present invention are advantageous in that they are non-toxic, their effects are reversible, they are devoid of endotoxin contamination, readily synthesized and inexpensive to produce and purify.

There remains a need in the art for materials and methods to modulate immune responses and to facilitate the delivery of therapeutic agents. This need and others are met by the present invention.

### SUMMARY OF THE INVENTION

The present invention provides novel peptide conjugates. Such peptide conjugates of the invention may be of any length. In some embodiments, peptide conjugates according to the invention may comprise a peptide from three to ten amino acids in length. In some embodiments, peptide conjugates of the invention may comprise, consist essentially of, or consist of a peptide that comprises, consists essentially of, or consists of an amino acid sequence selected from the group consisting of SEQ ID NOs: 1-291. In some embodiments, peptide conjugates of the invention may comprise, consist essentially of, or consist of a peptide that comprises, consists essentially of, or consists of an amino acid sequence selected from the group consisting of SEQ ID NOs:1, 2, 6, 7, 10, 11, 12, 13, 14, 17, 18, 19, 20, 22, 23, 24, 25, 26, 29, 30, 32, 35, 51, 63, 74, 75, 76, 78, 80, 83, 87, 96, 97, 101, 114, 115, 118, 119, 121, 125, 126, 127, 131, 132, 135, 136, 138, 141, 145, 148, 150, 154, 157, 159, 160, 161, 162, 168. 183, 196, 204, 206, 207, 208, 210, 211 and 223.

In certain embodiments, peptide conjugates of the invention may comprise a linker. The linker region can comprise polyethers, hydrocarbons, aromatic rings, heterocyclic rings, amino acid residues, or substituted or unsubstituted hydrocarbon chains useful for connecting a peptide selected from the group consisting of SEQ ID NOs: 1-291 with an additional active agent. In some embodiments, the linker region can comprise polyethers, hydrocarbons, aromatic rings, heterocyclic rings, amino acid residues, or substituted or unsubstituted hydrocarbon chains useful for connecting a peptide having an amino acid sequence selected from the group consisting of SEQ ID NOs:1, 2, 6, 7, 10, 11, 12, 13, 14, 17, 18, 19, 20, 22, 23, 24, 25, 26, 29, 30, 32, 35, 51, 63, 74, 75, 76, 78, 80, 83, 87, 96, 97, 101, 114, 115, 118, 119, 121, 125, 126, 127, 131, 132, 135, 136, 138, 141, 145, 148, 150, 154, 157, 159, 160, 161, 162, 168. 183, 196, 204, 206, 207, 208, 210, 211 and 223 with an additional active agent. The linker region may also connect the peptide to the additional active agent via the formation of other types of covalent bonds including thioether, ether, ester, thioester, sulfone, and phosphate bonds, depending on the structures of the linker region and the additional active agent.

The linker region can be designed to be non-functional or functional. “Non-functional” refers to non-reactive hydrocarbon chains, simple amino acid sequences, or other sequences that simply bind covalently to the peptide residues on one end and the additional active agent (“cargo molecule”) on the other end. A “functional linker” can comprise amino acid residues that confer biological properties useful for imaging, diagnostics, therapy, etc. Such a functionality could include peptide or protein binding motifs, protein kinase consensus sequences, protein phosphatase consensus sequences, or protease-reactive or protease-specific sequences. Protease sequences are particularly useful as they will result in amplification of an imaging, radiotherapeutic, diagnostic, or therapeutic effect through enzymatic action on the conjugate complex, thereby increasing the localized concentration of a cleaved and subsequently trapped cargo molecule such as a therapeutic agent. Another suitable functional linker is a Ca-responsive protein domain such as an EF-hand domain. A Ca-responsive domain renders the complex responsive to an intracellular signaling cascade by changing conformation and activity in response to a second messenger, thereby changing activity of the complex.

The present invention also provides compositions, e.g., pharmaceutical compositions, comprising one or more peptide conjugates of the invention. Peptide conjugates for use in compositions of the invention may comprise a peptide of any length. In some embodiments, such peptide conjugates may comprise a peptide of between three to ten amino acids in length. Suitable peptide conjugates for use in the compositions of the invention include, but are not limited to, peptide conjugates comprising peptides that comprise, consist essentially of, or consist of an amino acid sequence selected from the group consisting of SEQ ID NOs:1-291. In some embodiments, peptide conjugates for use in the compositions of the invention include, but are not limited to, peptide conjugates comprising peptides that comprise, consist essentially of, or consist of an amino acid sequence selected from the group consisting of SEQ ID NOs:1, 2, 6, 7, 10, 11, 12, 13, 14, 17, 18, 19, 20, 22, 23, 24, 25, 26, 29, 30, 32, 35, 51, 63, 74, 75, 76, 78, 80, 83, 87, 96, 97, 101, 114, 115, 118, 119, 121, 125, 126, 127, 131, 132, 135, 136, 138, 141, 145, 148, 150, 154, 157, 159, 160, 161, 162, 168. 183, 196, 204, 206, 207, 208, 210, 211 and 223.

Compositions of the invention may further comprise one or more additional active agents. Typically additional active agents may be therapeutic agents, imaging agents, and/or immunogenic agents. Examples of suitable additional therapeutic agents include, but are not limited to, glucose metabolism agents (e.g., insulin), antibiotics, antineoplastics, antihypertensives, antiepileptics, central nervous system agents, and immune system suppressants. Examples of suitable additional immunogenic agents include, but are not limited to, antigens. Suitable additional imaging agents include, but are not limited to, agents comprising one or more radioactive atoms. A pharmaceutical composition of the invention may comprise one or more pharmaceutically acceptable excipients.

Compositions of the invention, for example, pharmaceutical compositions, may be formulated for any type of delivery. For example, compositions of the invention may be formulated for intestinal delivery, e.g., may be delayed release compositions. Compositions of the invention may also be formulated for pulmonary delivery, oral delivery and/or transcutaneous delivery.

In one embodiment, the present invention provides a method of treating a disease in a subject in need thereof. Methods of the invention may comprise administering to the subject a pharmaceutical composition comprising one or more peptide conjugates of the invention. Methods of the invention may comprise administering to the subject a pharmaceutical composition comprising one or more peptide conjugates and one or more additional therapeutic agents. In one embodiment, the present invention provides a method of treating diabetes in a subject in need thereof. In another embodiment, the present invention provides a method of treating an excessive or undesirable immune response in a subject in need thereof. In another embodiment, the present invention provides a method of treating inflammation in a subject in need thereof. In specific embodiments, the present invention provides methods of treating inflammatory bowel disease in a subject in need thereof. Inflammatory bowel disease that can be treated using methods of the present invention may be Crohn's disease or ulcerative colitis. In another embodiment, the present invention provides methods of treating cancer in a subject in need thereof.

In certain embodiments, pharmaceutical compositions of the present invention may comprise one or more nucleic acid molecules including, for example, siRNAs. In other embodiments, pharmaceutical compositions of the present invention may comprise one or more insulins and/or derivatives thereof. In other embodiments, pharmaceutical compositions of the present invention may comprise one or more anti-inflammatory agents. In other embodiments, pharmaceutical compositions of the present invention may comprise one or more immune-suppressive drugs, for example, cyclosporin A. In another embodiment, pharmaceutical compositions of the present invention may comprise one or more anticancer agents.

### BRIEF DESCRIPTION OF THE DRAWINGS

FIG. 1 is a schematic showing the steps involved in solid phase synthesis of an exemplary permeability inducer of the invention.

FIG. 2 is a schematic showing the architecture of a novel peptide conjugate of the invention.

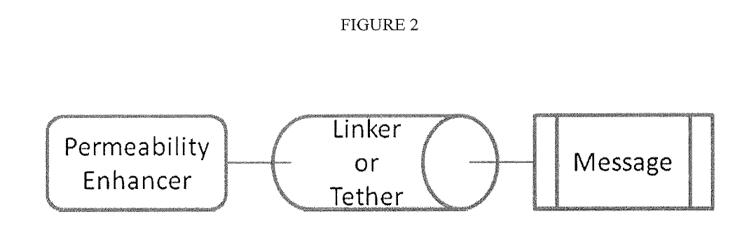

FIG. 3 is a schematic showing the attachment of linkers via the N- and C-termini of SEQ ID NO:1. X=an additional active agent.

FIG. 4 is a schematic showing the attachment of linkers via amino acid side chains at positions 1 (F/Phe) of SEQ ID NO:1. X=an additional active agent.

FIG. 5 is a schematic showing the attachment of linkers via amino acid side chains at positions 2 (C/Cys) of SEQ ID NO:1. X=an additional active agent.

FIG. 6 is a schematic showing the attachment of linkers via amino acid side chains at positions 5 (R/Arg) of SEQ ID NO:1. X=an additional active agent.

FIG. 7 is a schematic showing peptide conjugates with linker attachment via the N- and C-termini of SEQ ID NO:183.

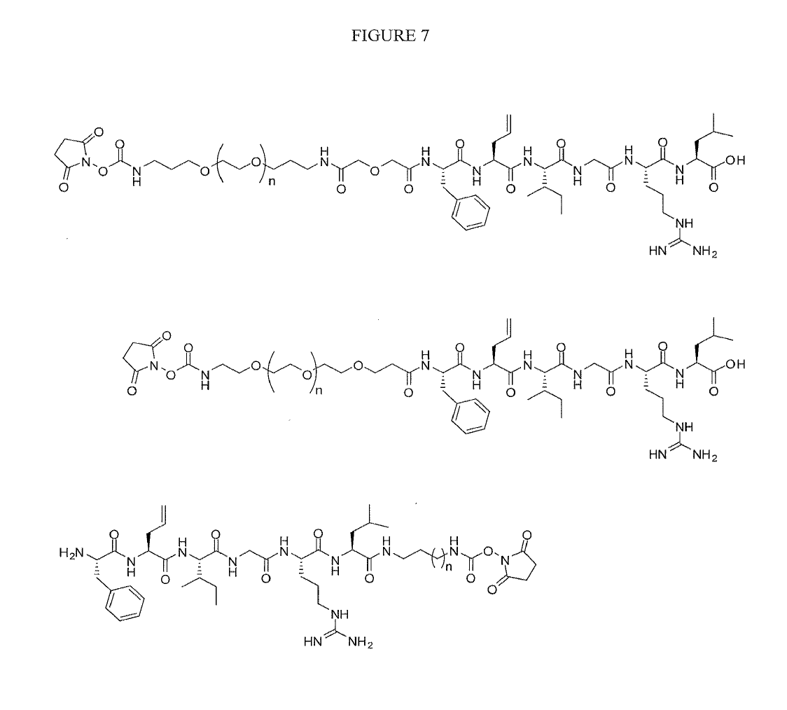

FIG. 8 is a bar graph showing the results of a Real-Time Cell Electronic Sensing (ACEA) assay comparing the activity of various peptides to known peptide permeability inducer FCIGRL.

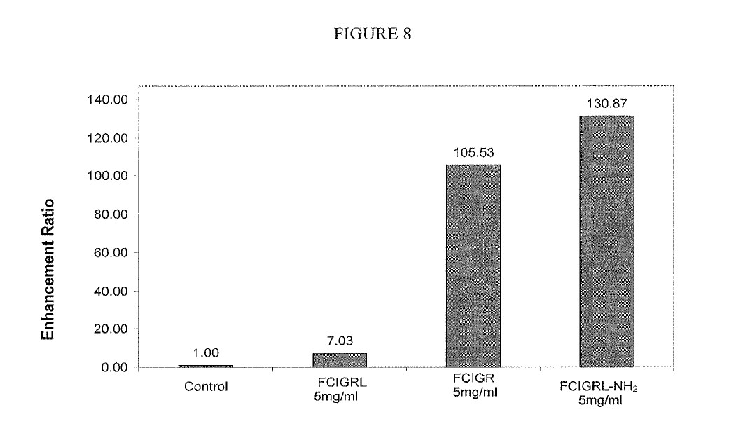

FIG. 9 is a fluorescence microscopy analysis of the effects of peptide permeability inducer FCIGRL on IEC6 cells grown in monolayer and stained for F-actin.

FIG. 10 is a fluorescence microscopy analysis of the effects of PT-gliadin, peptide permeability inducer FCIGRL, and various doses of peptide permeability inducer FCIGR on IEC6 cells grown in monolayer and stained for F-actin.

FIG. 11 is a fluorescence microscopy analysis of the effects of peptide permeability inducer FCIGRL on Caco-2 cells grown in monolayer and stained for tight junction protein ZO-1.

FIG. 12 is a fluorescence microscopy analysis of the effects of peptide permeability inducer FCIGR on Caco-2 cells grown in monolayer and stained for tight junction protein ZO-1.

### DETAILED DESCRIPTION OF THE INVENTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

### Permeability Inducers

As used herein, a “permeability inducer” is a compound that mediates or facilitates or augments an increase in the permeability of a biological barrier, and such “permeability inducers” may also be “tight junction agonists” that mediate or facilitate or augment the physiological, transient opening of tight junctions, for example, the tight junctions between adjacent epithelial cells. Such “permeability inducers” may also be “permeability enhancers” that enhance or supplement or augment an increase in the permeability of a biological barrier. An example of a permeability inducer is zonula occludens toxin (ZOT), which is produced by Vibrio cholerae. ZOT is also a tight junction agonist. A ZOT receptor agonist is a compound which is believed to mediate tight junction opening through the same receptor utilized by ZOT. In some embodiments, a permeability inducer may comprise a peptide.

As used herein a subject is any animal, e.g., mammal, upon which methods of the invention may be practiced and/or to which materials of the present invention may be administered. Subjects include, but are not limited to, humans.

Peptide conjugates of the invention may comprise peptide permeability inducers. An exemplary peptide permeability inducer is a peptide that comprises the amino acid sequence Phe-Cys-Ile-Gly-Arg-Leu (FCIGRL; SEQ ID NO:1). In certain embodiments the permeability inducer comprises a salt of a peptide that comprises the amino acid sequence Phe-Cys-Ile-Gly-Arg-Leu (FCIGRL; SEQ ID NO:1). Peptide salts of the invention are well-known to those of skill in the art. Additional examples of peptide permeability inducers of the invention include, but are not limited to, peptides wherein one or more amino acids of SEQ ID NO:1 have been substituted with a different amino acid. In some embodiments, only one position of SEQ ID NO:1 will be substituted. Substitutions may be made at any position of SEQ ID NO:1. In some embodiments, substitutions may be made at positions 1, 2, 3, 4, 5, or 6 of SEQ ID NO:1. In some embodiments, a peptide permeability inducer may comprise one or more D-amino acids.

Throughout the present specification peptides are represented in accordance with the conventional manner, that is, reading from N-terminus (amino terminus) at the left to the C-terminus (carboxyl terminus) at the right. In the peptide conjugates of the present invention including those containing the amino acid sequence of SEQ ID NO:1, the C-terminus may be any of a carboxyl group (—COOH), a carboxylate (—000), an amide (—CONH₂) and an ester (—COOR).

Examples of the ester group shown by R include a C₁₋₆ alkyl group such as methyl, ethyl, n-propyl, isopropyl, n-butyl, etc.; a C₃₋₈ cycloalkyl group such as cyclopentyl, cyclohexyl, etc.; a C₆₋₁₂ aryl group such as phenyl, α-naphthyl, etc.; a C₇₋₁₄ aralkyl group such as a phenyl-C₁₋₂ alkyl group, e.g., benzyl, phenethyl, etc.; an α-naphthyl-C₁₋₂ alkyl group such as α-naphthylmethyl, etc.; and the like. In addition, pivaloyloxymethyl or the like, which is used widely as an ester for oral administration, may also be used.

Where the peptide/protein of the invention contains a carboxyl group (or a carboxylate) at a position other than the C-terminus, it may be amidated or esterified and such an amide or ester is also included within the peptide/protein of the invention. The ester group may be the same group as that described with respect to the above C-terminal ester.

Furthermore, examples of the peptide/protein of the invention include variants of the above peptide/protein, wherein the amino group at the N-terminus (e.g., methionine residue) of the peptide is protected with a protecting group (e.g., a. C₁₋₆ acyl group such as a C₂₋₆ alkanoyl group, e.g., formyl group, acetyl group, etc.); those wherein the N-terminal region is cleaved in vivo and the glutamyl group thus formed is pyroglutaminated; those wherein a substituent (e.g., —OH, —SH, amino group, imidazole group, indole group, guanidino group, etc.) on the side chain of an amino acid in the molecule is protected with a suitable protecting group (e.g., a C₁₋₆ acyl group such as a C₂₋₆ alkanoyl group, e.g., formyl group, acetyl group, etc.), or conjugated peptides/conjugated proteins such as glycopeptides/glycoproteins having sugar chains.

When the permeability inducer is a peptide, any length of peptide may be used. Generally, the size of the peptide permeability inducer will range from about 3 to about 100, from about 3 to about 90, from about 3 to about 80, from about 3 to about 70, from about 3 to about 60, from about 3 to about 50, from about 3 to about 40, from about 3 to about 30, from about 3 to about 25, from about 3 to about 20, from about 3 to about 15, from about 3 to about 10, from about 3 to about 9, from about 3 to about 8, from about 3 to about 7, from about 3 to about 6, from about 3 to about 5, or from about 3 to about 4 amino acids in length. As used herein, “about” used to modify a numerical value means within 10% of the value. Peptide permeability inducers of the invention may be 3, 4, 5, 6, 7, 8, 9, or 10 amino acids in length.

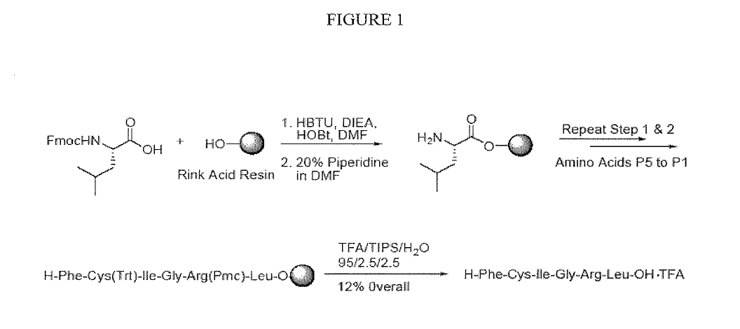

The peptide permeability inducers can be chemically synthesized and purified using well-known techniques, such as described in High Performance Liquid Chromatography of Peptides and Proteins: Separation Analysis and Conformation, Eds. Mant et al., C.R.C. Press (1991), and a peptide synthesizer, such as Symphony (Protein Technologies, Inc); or by using recombinant DNA techniques, i.e., where the nucleotide sequence encoding the peptide is inserted in an appropriate expression vector, e.g., an E. coli or yeast expression vector, expressed in the respective host cell, and purified there from using well-known techniques. A schematic representation of a solid phase synthesis of an exemplary permeability inducer of the invention is shown in FIG. 1.

### Compositions

Typically, compositions, such as pharmaceutical compositions, comprise a peptide conjugate comprising a peptide permeability inducer and optionally one or more additional active agents. Peptide permeability inducers may be present in an amount sufficient to facilitate the transportation of one or more additional active agents across a cell membrane; in an amount sufficient to facilitate the opening of tight junctions, for example, the tight junctions between adjacent epithelial cells; or in amount sufficient to modulate an immune response to an antigen; or in an amount sufficient to reduce inflammation, in a subject in need thereof. The amount of peptide permeability inducer employed in any given composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation.

Generally, a pharmaceutical composition of the invention will comprise an amount of peptide conjugate in the range of about 1 μg to 1 g, preferably about 1 mg to about 1000 mg, or from about 10 mg to about 100 mg, or from about 10 mg to about 50 mg, or from about 10 mg to about 25 mg of peptide conjugate.

Compositions of the invention may comprise one or more peptide conjugates at a level of from about 0.1 wt % to about 20 wt %, from about 0.1 wt % to about 18 wt %, from about 0.1 wt % to about 16 wt %, from about 0.1 wt % to about 14 wt %, from about 0.1 wt % to about 12 wt %, from about 0.1 wt % to about 10 wt %, from about 0.1 wt % to about 8 wt %, from about 0.1 wt % to about 6 wt %, from about 0.1 wt % to about 4 wt %, from about 0.1 wt % to about 2 wt %, from about 0.1 wt % to about 1 wt %, from about 0.1 wt % to about 0.9 wt %, from about 0.1 wt % to about 0.8 wt %, from about 0.1 wt % to about 0.7 wt %, from about 0.1 wt % to about 0.6 wt %, from about 0.1 wt % to about 0.5 wt %, from about 0.1 wt % to about 0.4 wt %, from about 0.1 wt % to about 0.3 wt %, or from about 0.1 wt % to about 0.2 wt % of the total weight of the composition. As used herein, “about” used to modify a numerical value means within 10% of the value. Compositions of the invention may comprise one or more peptide conjugates at a level of about 0.1 wt %, about 0.2 wt %, about 0.3 wt %, about 0.4 wt %, about 0.5 wt %, about 0.6 wt %, about 0.7 wt %, about 0.8 wt %, or about 0.9 wt % based on the total weight of the composition.

Compositions of the invention may comprise one or more peptide conjugates at a level of from about 1 wt % to about 20 wt %, from about 1 wt % to about 18 wt %, from about 1 wt % to about 16 wt %, from about 1 wt % to about 14 wt %, from about 1 wt % to about 12 wt %, from about 1 wt % to about 10 wt %, from about 1 wt % to about 9 wt %, from about 1 wt % to about 8 wt %, from about 1 wt % to about 7 wt %, from about 1 wt % to about 6 wt %, from about 1 wt % to about 5 wt %, from about 1 wt % to about 4 wt %, from about 1 wt % to about 3 wt %, or from about 1 wt % to about 2 wt % of the total weight of the composition. As used herein, “about” used to modify a numerical value means within 10% of the value. Compositions of the invention may comprise one or more peptide conjugates at a level of about 1 wt %, about 2 wt %, about 3 wt %, about 4 wt %, about 5 wt %, about 6 wt %, about 7 wt %, about 8 wt %, or about 9 wt % based on the total weight of the composition.

Compositions of the invention, for example, pharmaceutical compositions comprising one or more peptide conjugates and optionally one or more additional active agents, may be formulated for pulmonary delivery (e.g., may be pulmonary dosage forms). Typically such compositions may be provided as pharmaceutical aerosols, e.g., solution aerosols or powder aerosols. Those of skill in the art are aware of many different methods and devices for the formation of pharmaceutical aerosols, for example, those disclosed by Sciarra and Sciarra, Aerosols, in Remington: The Science and Practice of Pharmacy, 20th Ed., Chapter 50, Gennaro et al. Eds., Lippincott, Williams and Wilkins Publishing Co., (2000).

In one embodiment, the dosage forms are in the form of a powder aerosol (i.e, comprise particles). These are particularly suitable for use in inhalation delivery systems. Powders may comprise particles of any size suitable for administration to the lung.

Powder formulations may optionally contain at least one particulate pharmaceutically acceptable carrier known to those of skill in the art. Examples of suitable pharmaceutical carriers include, but are not limited to, saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. In one embodiment, a powder formulation may comprise lactose as a carrier.

Powder formulations may be contained in any container known to those in the art. Containers may be capsules of, for example, gelatin or plastic, or in blisters (e.g. of aluminum or plastic), for use in a dry powder inhalation device. In some embodiments, the total weight of the formulation in the container may be from about 5 mg to about 50 mg. In other embodiments, powder formulations may be contained in a reservoir in a multi-dose dry powder inhalation device adapted to deliver a suitable amount per actuation.

Powder formulations typically comprise small particles. Suitable particles can be prepared using any means known in the art, for example, by grinding in an airjet mill, ball mill or vibrator mill, sieving, microprecipitation, spray-drying, lyophilisation or controlled crystallisation. Typically, particles will be about 10 microns or less in diameter. Particles for use in the compositions of the invention may have a diameter of from about 0.1 microns to about 10 microns, from about 0.1 microns to about 9 microns, from about 0.1 microns to about 8 microns, from about 0.1 microns to about 7 microns, from about 0.1 microns to about 6 microns, from about 0.1 microns to about 5 microns, from about 0.1 microns to about 4 microns, from about 0.1 microns to about 3 microns, from about 0.1 microns to about 2 microns, from about 0.1 microns to about 1 micron, from about 0.1 microns to about 0.5 microns, from about 1 micron to about 10 microns, from about 1 micron to about 9 microns, from about 1 micron to about 8 microns, from about 1 micron to about 7 microns, from about 1 micron to about 6 microns, from about 1 micron to about 5 microns, from about 1 micron to about 4 microns, from about 1 micron to about 3 microns, from about 1 micron to about 2 microns, from about 2 microns to about 10 microns, from about 2 microns to about 9 microns, from about 2 microns to about 8 microns, from about 2 microns to about 7 microns, from about 2 microns to about 6 microns, from about 2 microns to about 5 microns, from about 2 microns to about 4 microns, or from about 2 microns to about 3 microns. As used herein, “about” used to modify a numerical value means within 10% of the value. In some embodiments, particles for use in the invention may be about 1 micron, about 2 microns, about 3 microns, about 4 microns, about 5 microns, about 6 microns, about 7 microns, about 8 microns, about 9 microns, or about 10 microns in diameter.

In one embodiment, the dosage forms are in the form of a solution aerosol (i.e., comprise droplets). Typically, droplets will be about 10 microns or less in diameter. Droplets for use in the compositions of the invention may have a diameter of from about 0.1 microns to about 10 microns, from about 0.1 microns to about 9 microns, from about 0.1 microns to about 8 microns, from about 0.1 microns to about 7 microns, from about 0.1 microns to about 6 microns, from about 0.1 microns to about 5 microns, from about 0.1 microns to about 4 microns, from about 0.1 microns to about 3 microns, from about 0.1 microns to about 2 microns, from about 0.1 microns to about 1 micron, from about 0.1 microns to about 0.5 microns, from about 1 micron to about 10 microns, from about 1 micron to about 9 microns, from about 1 micron to about 8 microns, from about 1 micron to about 7 microns, from about 1 micron to about 6 microns, from about 1 micron to about 5 microns, from about 1 micron to about 4 microns, from about 1 micron to about 3 microns, from about 1 micron to about 2 microns, from about 2 microns to about 10 microns, from about 2 microns to about 9 microns, from about 2 microns to about 8 microns, from about 2 microns to about 7 microns, from about 2 microns to about 6 microns, from about 2 microns to about 5 microns, from about 2 microns to about 4 microns, or from about 2 microns to about 3 microns. As used herein, “about” used to modify a numerical value means within 10% of the value. In some embodiments, particles and/or droplets for use in the invention may be about 1 micron, about 2 microns, about 3 microns, about 4 microns, about 5 microns, about 6 microns, about 7 microns, about 8 microns, about 9 microns, or about 10 microns in diameter.

The compositions of the invention may be formulated for enteric delivery, for example, may comprise one or more coatings including, for example, a delayed release coating containing one or more enteric agents. A delayed release coating is typically substantially stable in gastric fluid and substantially unstable (e.g., dissolves rapidly or is physically unstable) in intestinal fluid, thus providing for substantial release of the peptide conjugate and/or additional active agent from the composition in the duodenum or the jejunum.

The term “stable in gastric fluid” refers to a composition that releases 30% or less by weight of the total peptide conjugate and/or additional active agent in the composition in gastric fluid with a pH of 5 or less, or simulated gastric fluid with a pH of 5 or less, in approximately sixty minutes. Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

Compositions of the of the invention may release from about 0% to about 30%, from about 0% to about 25%, from about 0% to about 20%, from about 0% to about 15%, from about 0% to about 10%, from about 5% to about 30%, from about 5% to about 25%, from about 5% to about 20%, from about 5% to about 15%, from about 5% to about 10% by weight of the total peptide conjugate and/or additional active agent in the composition in gastric fluid with a pH of 5 or less, or simulated gastric fluid with a pH of 5 or less, in approximately sixty minutes. As used herein, “about” used to modify a numerical value means within 10% of the value. Compositions of the invention may release about 1%, about 2%, about 3%, about 4%, about 5%, about 6%, about 7%, about 8%, about 9%, or about 10% by weight of the total peptide conjugate in the composition in gastric fluid with a pH of 5 or less, or simulated gastric fluid with a pH of 5 or less, in approximately sixty minutes.

The term “unstable in intestinal fluid” refers to a composition that releases 70% or more by weight of the total peptide conjugate and/or additional active agent in the composition in intestinal fluid or simulated intestinal fluid in approximately sixty minutes. The term “unstable in near neutral to alkaline environments” refers to a composition that releases 70% or more by weight of the total amount of peptide conjugate and/or additional active agent in the composition in intestinal fluid with a pH of 5 or greater, or simulated intestinal fluid with a pH of 5 or greater, in approximately ninety minutes. For example, a composition that is unstable in near neutral or alkaline environments may release 70% or more by weight of a peptide conjugate and/or additional active agent in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 60 minutes, or from about 30 minutes to about 60 minutes.

Compositions of the invention may be formulated for transcutaneous delivery (e.g., may be transcutaneous dosage forms). Typically such compositions may be provided as topical solutions and/or gels. Those of skill in the art are aware of many different methods and devices for the formation of topical medications, for example, those disclosed by Block, Medicated Topicals, in Remington: The Science and Practice of Pharmacy, 20th Ed., Chapter 44, Gennaro et al. Eds., Lippincott, Williams and Wilkins Publishing Co., (2000).

### Additional Active Agents

In addition to one or more peptide conjugates, compositions of the invention may further comprise one or more additional active agents, e.g., therapeutic agents, immunogenic agents and/or imaging agents.

Additional therapeutic agents that can be used in the compositions of the invention include agents that act on any organ of the body, such as heart, brain, intestine, or kidneys. Suitable additional therapeutic agents include, but are not limited to, glucose metabolism agents (e.g., insulin), antibiotics, antineoplastics, antihypertensives, antiepileptics, central nervous system agents, anti-inflammatory agents and immune system suppressants.

Additional therapeutic agents that can be used in the compositions of the invention include immunosuppressive agents. Such immunosuppressants used in the method and composition of the invention can be any agent which tends to attenuate the activity of the humoral or cellular immune systems. In particular, in one aspect the invention comprises compositions wherein the immunosuppressant is selected from the group consisting of cyclosporin A, FK506, prednisone, methylprednisolone, cyclophosphamide, thalidomide, azathioprine, and daclizumab, physalin B, physalin F, physalin G, seco-steroids purified from Physalis angulata L., 15-deoxyspergualin (DSG, 15-dos), MMF, rapamycin and its derivatives, CCI-779, FR 900520, FR 900523, NK86-1086, depsidomycin, kanglemycin-C, spergualin, prodigiosin25-c, cammunomicin, demethomycin, tetranactin, tranilast, stevastelins, myriocin, gliooxin, FR 651814, SDZ214-104, bredinin, WS9482, mycophenolic acid, mimoribine, misoprostol, OKT3, anti-IL-2 receptor antibodies, azasporine, leflunomide, mizoribine, azaspirane (SKF 105685), paclitaxel, altretamine, busulfan, chlorambucil, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea, and combinations thereof. In one more particular aspect, the immunosuppressant is cyclosporin A.

Furthermore, the additional therapeutic agent can be selected from the group consisting of a chemotherapeutic, a gene therapy vector, a growth factor, a contrast agent, an angiogenesis factor, a radionuclide, an anti-infection agent, an anti-tumor compound, a receptor-bound agent, a hormone, a steroid, a protein, a complexing agent, a polymer, a thrombin inhibitor, an antithrombogenic agent, a tissue plasminogen activator, a thrombolytic agent, a fibrinolytic agent, a vasospasm inhibitor, a calcium channel blocker, a nitrate, a nitric oxide promoter, a vasodilator, an antihypertensive agent, an antimicrobial agent, an antibiotic, a glycoprotein IIb/IIIa inhibitor, an inhibitor of surface glycoprotein receptors, an antiplatelet agent, an antimitotic, a microtubule inhibitor, a retinoid, an antisecretory agent, an actin inhibitor, a remodeling inhibitor, an antisense nucleotide, an agent for molecular genetic intervention, an antimetabolite, an antiproliferative agent, an anti-cancer agent, a dexamethasone derivative, an anti-inflammatory steroid, a non-steroidal anti-inflammatory agent, an immunosuppressive agent, a PDGF antagonist, a growth hormone antagonist, a growth factor antibody, an anti-growth factor antibody, a growth factor antagonist, a dopamine agonist, a radiotherapeutic agent, an iodine-containing compound, a barium-containing compound, a heavy metal functioning as a radiopaque agent, a peptide, a protein, an enzyme, an extracellular matrix component, a cellular component, an angiotensin converting enzyme inhibitor, a 21-aminosteroid, a free radical scavenger, an iron chelator, an antioxidant, a sex hormone, an antipolymerase, an antiviral agent, an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents, gene therapy agents, a prodrug, a photodynamic therapy agent, and an agent for treating benign prostatic hyperplasia (BHP), a ¹⁴C-, ³H-¹³¹I-, ³²P- or ³⁶S-radiolabelled form or other radiolabelled form of any of the foregoing, and combinations thereof.

More particularly, the additional therapeutic agent can be selected from the group consisting of parathyroid hormone, heparin, human growth hormone, covalent heparin, hirudin, hirulog, argatroban, D-phenylalanyl-L-poly-L-arginyl chloromethyl ketone, urokinase, streptokinase, nitric oxide, triclopidine, aspirin, colchicine, dimethyl sulfoxide, cytochalasin, deoxyribonucleic acid, methotrexate, tamoxifen citrate, dexamethasone, dexamethasone sodium phosphate, dexamethasone acetate, cyclosporin, trapidal, angiopeptin, angiogenin, dopamine, ⁶⁰Co, ¹⁹²Ir, ³²P, ¹¹¹In, ⁹⁰Y, ^{99m}Tc, pergolide mesylate, bromocriptine mesylate, gold, tantalum, platinum, tungsten, captopril, enalapril, ascorbic acid, α-tocopherol, superoxide dismutase, deferoxamine, estrogen, azidothymidine (AZT), acyclovir, famciclovir, rimantadine hydrochloride, ganciclovir sodium, 5-aminolevulinic acid, meta-tetrahydroxyphenylchlorin, hexadecafluoro zinc phthalocyanine, tetramethyl hematoporphyrin, and rhodamine 123, and combinations thereof.

Compositions of the invention may comprise one or more nucleic acids, for example, deoxyribonucleic acid (DNA) or ribonucleic acid (RNA). Examples of such nucleic acids that can be used in the compositions of the invention (e.g., therapeutic compositions) include oligonucleotides and polynucleotides, complementary DNA sequences encoding one or more functional peptides or polypeptides, messenger RNA sequences encoding one or more functional peptides or polypeptides, small nucleic acid molecules, such as short interfering nucleic acid (siNA), short interfering RNA (siRNA), double-stranded RNA (dsRNA), micro-RNA (miRNA), and short hairpin RNA (shRNA) molecules, and inibitors thereof (RNAi inhibitors); and miRNA inhibitors, nucleic acids, polynucleotides and oligonucleotides. Other nucleic acid compounds as are known in the art can also be used.

In particular embodiments, the compositions of the invention comprise siRNA molecules 19 to 30 nucleotides in length. In further particular embodiments, the compositions of the invention comprise siRNA molecules 19 to 23 nucleotides in length. In specific embodiments of the invention, the siRNA molecule is 19, 20, 21, 22, 23, 24, 25, 26, 27, 28, 29 or 30 nucleotide residues in length.

In some embodiments, the compositions of the invention comprise siRNA molecules that decrease or down-regulate expression of, or can be originated from, a gene encoding another additional active agent, an oncogene, a cytokine, an interleukin, a chemokine, a growth factor, a hormone, a tumor necrosis factor ligand (TNF), a human tumor necrosis factor receptor (TNFR), a G-protein coupled receptor, a receptor tyrosine kinase, an integrin, a toll-like receptor, an ion channel, or an enzyme.

Oncogenes whose expression can be decreased or down-regulated by compositions of the invention include, but are not limited to, c-myc, c-myb, c-fos, c-jun, c-raf, c-src or bcl-2.

Cytokines whose expression can be decreased or down-regulated by compositions of the invention include, but are not limited to, GM-CSF, G-CSF, IFN-alpha, IFN-beta or IFN-gamma.

Interleukins whose expression can be decreased or down-regulated by compositions of the invention include, but are not limited to, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21 or IL-22.

Chemokines whose expression can be decreased or down-regulated by compositions of the invention include, but are not limited to, XCL1, XCL2, CX₃CL1, CCL1, CCL2, CCL3, CCL4, CCL5, CCL6, CCL7, CCL8, CCL9/10, CCL11, CCL12, CCL13, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15 or CXCL16.

Growth factors whose expression can be decreased or down-regulated by compositions of the invention include, but are not limited to, fibroblast growth factors (FGF) such as, for example, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15; vascular endothelial growth factor (VEGF), VEGF-2, VEGF-B, VEGF-C, VEGF-D, VEGF-E; platelet derived growth factor (PDGF) PDGF-A, PDGF-B, PDGF-C and PDGF-D; Glioma Derived Growth Factor (GDGF); Placental Growth Factor (PIGF); or Placental Growth Factor-2 (PIGF-2).

TNF ligands and receptors whose expression can be decreased or down-regulated by compositions of the invention include, but are not limited to, TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, AIM-I, AIM-II, APRIL, endokine-alpha, TR6, OPG, and neutrokine-alpha, OX40, nerve growth factor (NGF), Fas, CD30, CD27, CD40 and 4-IBB, TR2, DR3, DR4, TR2, TR6, TR7, TRANK, TR9, TR10, 312C2, TR12, CD154, CD70 or CD153.

G protein-coupled receptors whose expression can be decreased or down-regulated by compositions of the invention include, but are not limited to, 5-hydroxytryptamine (serotonin) receptors such as 5-HT_{1A}, 5-HT_{1B}, 5-HT_{1D}, 5-HT_{1E}, 5-HT_{1F}, 5-HT_{2A}, 5-HT_{2B}, 5-HT_{2C}, 5-HT₄, 5-HT_{5A}, 5-HT₆, and 5-HT₇; acetylcholine receptors such as m1AchR, m2AchR, m3AchR, m4AchR, and m5AchR; adrenergic receptors such as α_{1A}-adrenoceptor, α_{1B}-adrenoceptor, α_{1D}-adrenoceptor, α_{2A}-adrenoceptor, α_{2B}-adrenoceptor, α_{2C}-adrenoceptor, β₁-adrenoceptor, β₂-adrenoceptor, and β₃-adrenoceptor; adenosine receptors such as A₁ adenosine receptor, A_{2A} adenosine receptor, A_{2B} adenosine receptor and A₃ adenosine receptor; angiotensin receptors such as AT₁ receptor and AT₂ receptor; anaphylotoxin receptors such as C3a receptor, C5a receptor and C5L2 receptor; apelin receptor; bile acid receptor; bombesin receptors such as BB1, BB2 and BB3; bradykinin receptors such as B₁ receptor and B₂ receptor; calcitonin receptors such as CT, AMY₁, AMY₂, AMY₃, CALCRL, CGRP₁, AM₁, and AM₂; cannabinoid receptors such as CB₁ receptor and CB₂ receptor; calcium sensing receptor such as CaS, and GPRC₆; chemokine receptors such as CCR1, CCR2, CCR3, CCR4, CCR5, CCR6, CCR7, CCR8, CCR9, CCR10, CXCR1, CXCR2, CXCR3, CXCR4, CXCR5, CXCR6, CX₃CR1 and XCR1; cholecystokinin receptors such as CCK₁ and CCK₂; corticotropin-releasing factor receptors such as CRF₁ and CRF₂; Dopamine receptors such as D₁ receptor, D₂ receptor, D₃ receptor, D₄ receptor and D₅ receptor; Endothelin receptors such as ET_{A} receptor and ET_{B} receptor; Estrogen (G protein coupled) receptor such as GPER; Formylpeptide receptors such as FPR, FPRL1 and FPRL2; Frizzled receptors such as FZD₁, FZD₂, FZD₃, FZD₄, FZD₅, FZD₆, FZD₇, FZD₈, FZD₉, FZD₁₀ and SMO; Free fatty acid receptors such as FFA1, FFA2, FFA3 and GPR42; Galanin receptors such as GAL₁, GAL₂ and GAL₃; GABA_{B} receptors such as GABA_{B1}, GABA_{B2} and GABA_{B}; Ghrelin receptor; Glucagon receptor family members such as GHRH, GIP, GLP-1, GLP-2, glucagon and secretin; Gonadotrophin-releasing hormone receptors such as GnRH and GnRH2; GPRC5 receptors such as RAIG₁, RAIG₂, RAIG₃ and RAIG₄; Histamine receptors such as H₁, H₂, H₃ and H₄; KiSS1-derived peptide receptor; Leukotriene receptors such as ALX, BLT₁, BLT₂, CysLT₁, CysLT₂ and OXE; Lysophospholipid receptors such as LPA₁ receptor, LPA₂ receptor, LPA₃ receptor, S₁P₁ receptor, S₁P₂ receptor, S₁P₃ receptor, S₁P₄receptor and S₁P₅ receptor; Melatonin receptors such as MT₁ and MT₂; Melanocortin receptors such as MC₁, MC₂, MC₃, MC₄ and MC₅; Metabotropic glutamate receptors such as mGlu₁, mGlu₂, mGlu₃, mGlu₄, mGlu₅, mGlu₆, mGlu₇ and mGluₛ; Melanin-concentrating hormone receptors such as MCH₁ and MCH₂; Motilin receptor; Neurotensin receptors such as NTS₁ and NTS₂; Neuromedin U receptors such as NMU1 and NMU2; Neuropeptide S receptor; Neuropeptide Y receptors such as Y₁ receptor, Y₂ receptor, Y₄ receptor and Y₅ receptor; Neuropeptide W/neuropeptide B receptors such as NPBW1 and NPBW2; Neuropeptide FF/neuropeptide AF receptors such as NPFF1 and NPFF2; Nicotinic acid receptor such as GPR81, GPR109A and GPR109B; Opioid receptors such as δ-opioid receptor, κ-opioid receptor, μ-opioid receptor and NOP; Orexin receptors such as OX₁ and OX₂; P2Y receptors such as P2Y₁ receptor, P2Y₂ receptor, P2Y₄ receptor, P2Y₆ receptor, P2Y₁ᵢ receptor, P2Y₁₂ receptor, P2Y₁₃ receptor and P2Y₁₄ receptor; Parathyroid hormone receptors such as PTH1 and PTH2; Peptide P518 receptor QRFP; Platelet-activating factor receptor; Prostanoid receptors such as DP₁, DP₂, EP₁, EP₂, EP₃, EP₄, FP, IP₁ and TP; Prokineticin receptors such as PKR₁ and PKR₂; Protease-activated receptors such as PAR1, PAR2, PAR3 and PAR4; Prolactin-releasing peptide receptor; Relaxin family peptide receptors such as RXFP1, RXFP2, RXFP3 and RXFP4; Somatostatin receptors such as sst₁, sst₂, sst₃, sst₄ and sst₅; Tachykinin receptors such as NK₁, NK₂ and NK₃; Thyrotropin-releasing hormone receptor; Trace amine receptor; Urotensin receptor; Vasopressin and oxytocin receptors such as V_{1A}, V_{1B}, V₂ and OT; Vasoactive Intestinal Peptide (VIP) and PACAP receptors such as PAC₁, VPAC₁ and VPAC₂; class A orphan receptors such as CCRL2, CMKLR1, CMKOR1, EBI2, GPR1, GPR3, GPR4, GPR6, GPR12, GPR15, GPR17, GPR18, GPR19, GPR20, GPR21, GPR22, GPR23, GPR25, GPR26, GPR27, GPR31, GPR32, GPR33, GPR34, GPR35, GPR37, GPR37L₁, GPR39, GPR42, GPR45, GPR50, GPR52, GPR55, GPR61, GPR62, GPR63, GPR65, GPR68, GPR75, GPR78, GPR79, GPR82, GPR83, GPR84, GPR85, GPR87, GPR88, GPR92, GPR101, GPR119, GPR120, GPR132, GPR135, GPR139, GPR141, GPR142, GPR146, GPR148, GPR149, GPR150, GPR151, GPR152, GPR153, GPR160, GPR161, GPR162, GPR171, GPR173, GPR174, GPR182, LGR4, LGR5, LGR6, MAS1, MAS1L, MRGPRD, MRGPRE, MRGPRF, MRGPRG, MRGPRX1, MRGPRX2, MRGPRX3, MRGPRX4, OPN3, OPN5, OXGR1, P2RY5, P2RY8, P2RY10, SUCNR1, TAAR2, TAAR3, TAAR4, TAAR5, TAAR6, TAAR8 and TAAR9; class B orphan receptors such as BAI1, BAI2, BAI3, CD97, CELSR1, CELSR2, CELSR3, ELTD1, EMR1, EMR2, EMR3, EMR4, GPR56, GPR64, GPR97, GPR98, GPR110, GPR111, GPR112, GPR113, GPR114, GPR115, GPR116, GPR123, GPR124, GPR125, GPR126, GPR128, GPR133, GPR143, GPR144, GPR157, LPHN1, LPHN2 and LPHN3; Class C Orphan receptors such as GPR156, GPR158, GPR179, RAIG₁, RAIG₂, RAIG₃ and RAIG₄; or non-signalling 7TM chemokine-binding proteins such as CCPB2, CCRL1 and FY.

Receptor tyrosine kinases whose expression can be decreased or down-regulated by compositions of the invention include, but are not limited to, EGF receptors, Insulin receptors, PDGF receptors, FGF receptors, VEGF receptors, HGF receptors, Trk receptors, AXL receptors, LTK receptors, TIE receptors, ROR receptors, DDR receptors, KLG receptors, RYK receptors or MuSK receptors.

Integrins whose expression can be decreased or down-regulated by compositions of the invention include, but are not limited to, α1-β1 integrin, α2-β1 integrin, α3-β1 integrin, α4-β1 integrin, α5-β1 integrin, α6-β1 integrin, α7-β1 integrin, α8-β1 integrin, α9-β1 integrin, αV-β1 integrin, α4-β7 integrin, α6-β4 integrin, αD-β2 integrin, αL-β2 integrin, αM-β2 integrin, αV-β1 integrin, αV-β3 integrin, αV-β5 integrin, αV-β6 integrin, αV-β8 integrin, α-β2 integrin, αIIb-β3 integrin or αIELb-β7 integrin.

Toll-like receptors whose expression can be decreased or down-regulated by compositions of the invention include, but are not limited to, TLR1, TLR2, TLR3, TLR4, TLR5, TLR6, TLR7, TLR8, TLR9, TLR10, TLR11, TLR12 or TLR 13.

Ion channels whose expression can be decreased or down-regulated by compositions of the invention include, but are not limited to, any one or more subunit components of an extracellular ligand-gated ion channel such as the nicotine acetylcholine receptor (nAchR), the type A gamma-aminobutyric acid receptor (GABAAR), the glycine receptor (GlyR), the 5-hydroxytryptamine receptor (5-HT₃R), the ATP receptor (P2XR), the glutamate receptor (GluR) including AMPA, NMDA and kainite sensitive subtypes, and inward rectifying channels including ROMK, ROMK2, IRK, BIR, RACTK, Kir6.2, Kir1.1 and Kir6.1; a voltage-gated ion channel such as the sodium voltage-gated channels including mH1, mH2, SCN4A, PN1, PN3, SkM1, RSMK, Kat1, EAG, ELK, and Drk1, the potassium voltage-gated channels including Kv1.3, IKCa1, HERG, HCN, Kv, Kd, Kf, KCa, MaxiK, TASK-1, Shaker, Shal, Shab, Shaw, mink, IsK, KvLQT and KCNK, the calcium voltage-gated channels including L (HVA), T (LVA), N types and P types, the voltage-gated proton channels, the anion voltage-gated channel VDAC, I(h), I(f), I(Q), and I(AR); an intracellular ligand-gated ion channel such as the ATP-sensitive potassium channels (ATP-K) including ROMK2, IRK, BIR, RACTK, Kir, ADAC and ATP-regulated Potassium Channel, CFTR, the calcium-activated chloride channels (ClCas), ENaC, the ASIC family, the calcium-activated potassium channels including SK and BK, K(ATP), the cyclic nucleotide-gated channels (CNG Channels), the G-protein activated inwardly rectifying potassium channels including GIRK2 (Kir 3.2), the phosphoinositide-mediated calcium channel (IP(3) Receptor), the TRP (transient receptor potential) channels including TRP-PLIK, melastatin and MTR1, the calcium-activated chloride channels including SK3, the calcium release channels including P/Q type and L-type calcium channels, the ryanodine receptor (RyR), Icrac, CaT1, ECaC, SOC, ENaC, GORK, and the Aquaporins including AQP1; a mechanosensitive ion channel such as MscL, mink, IsK, VBAC, CCH1, Mid1, CLC chloride channels including CLC-O, CLC-1, CLC-2, GEF1, CLC-5, CLC-4, CLC-6, CLC-7, CLC-K1, CLC-K2, CLC-3, EriC and CLH-5, acid-sensing ion channels (ASICs) including ASIC3 and KCNK3; or a miscellaneous ion channel such as KCNK or a GAP junction composed of one or more connexins.

Compositions of the invention may comprise one or more immunogenic agents, for example, antigens. Examples of antigens that can be used in the compositions of the invention (e.g., immunogenic and/or vaccine compositions) include peptides, proteins, microorganisms (e.g., attenuated and/or recombinant microorganisms), cells (e.g., cancer cells and/or recombinant cells) and viruses (e.g., attenuated and/or recombinant viruses). Examples of peptide antigens include the B subunit of the heat-labile enterotoxin of enterotoxigenic E. coli, the B subunit of cholera toxin, capsular antigens of enteric pathogens, fimbriae or pili of enteric pathogens, HIV surface antigens, cancer antigens (e.g., cancer cells comprising antigens, isolated antigens, etc.), dust allergens, and acari allergens. Other immunogenic compounds as are known in the art can also be used.

Examples of attenuated microorganisms and viruses that can be used in the compositions of the invention (e.g., vaccine compositions) include those of enterotoxigenic Escherichia coli, enteropathogenic Escherichia coli, Vibrio cholerae, Shigella flexneri, Salmonella typhi and rotavirus (Fasano et al, In: Le Vaccinazioni in Pediatria, Eds. Vierucci et al, CSH, Milan, pages 109-121 (1991); Guandalini et al, In: Management of Digestive and Liver Disorders in Infants and Children, Elsevior, Eds. Butz et al, Amsterdam, Chapter 25 (1993); Levine et al, Sem. Ped. Infect. Dis., 5.243-250 (1994); and Kaper et al, Clin. Micrbiol. Rev., 8:48-86 (1995), each of which is incorporated by reference herein in its entirety).

Any antigen capable of inducing a protective immune response may be used in the vaccine compositions of the invention. Examples of suitable antigens include, but are not limited to, measles virus antigens, mumps virus antigens, rubella virus antigens, Corynebacterium diphtheriae antigens, Bordetella pertussis antigens, Clostridium tetani antigens, Bacillus anthracis antigens, Haemophilus influenzae antigens, smallpox virus antigens, and influenza virus antigens.

Compositions of the invention may further comprise one or more protease inhibitors. Any protease inhibitor can be used, including, but not limited to, a proteinase, peptidase, endopeptidase, or exopeptidase inhibitor. A cocktail of inhibitors can also be used. Alternatively, the protease inhibitors can be selected from the group consisting of bestatin, L-trans-3-carboxyoxiran-2-carbonyl-L-leucylagmatine, ethylenediaminetetraacetic acid (EDTA), phenylmethylsulfonylfluoride (PMSF), aprotinin, amyloid protein precursor (APP), amyloid beta precursor protein, α1-proteinase inhibitor, collagen VI, bovine pancreatic trypsin inhibitor (BPTI), 4-(2-aminoethyl)-benzenesulfonyl fluoride (AEBSF), antipain, benzamidine, chymostatin, ε-aminocaproate, N-ethylmaleimide, leupeptin, pepstatin A, phosphoramidon, and combinations thereof. Novel protease inhibitors can also be used. Indeed, protease inhibitors can be specifically designed or selected to decrease the proteolysis of the tight junction agonist and/or the therapeutic agent.

Compositions of the invention may also comprise one or more pharmaceutically acceptable excipients. Suitable excipients include, but are not limited to, buffers, buffer salts, bulking agents, salts, surface active agents, acids, bases, sugars, binders, and the like.

### Peptide Conjugate Linkers

The general structure of the peptide conjugates according to the present invention is based upon a novel combination of three components: 1) a peptide permeability inducer comprising D-amino acids and/or L-amino acids; 2) a functional or non-functional linker; and 3) one or more additional active agents, e.g., therapeutic agents, immunogenic agents and/or imaging agents. In particular embodiments the peptide conjugates comprise 1) a peptide permeability inducer having an amino acid sequence selected from the group consisting of SEQ ID NOs:1-291; 2) a functional or non-functional linker; and 3) one or more additional active agents, e.g., therapeutic agents, immunogenic agents and/or imaging agents.

The linker can be designed to be non-functional or functional. “Non-functional” refers to non-reactive hydrocarbon chains, simple amino acid sequences, or other sequences that simply bind covalently to the peptide permeability inducer on one end and the additional active agent on the other end. A “functional linker” can comprise amino acid residues that confer biological properties useful for imaging, diagnostics, therapy, etc. Such a functionality could include peptide or protein binding motifs, protein kinase consensus sequences, protein phosphatase consensus sequences, or protease-reactive or protease-specific sequences. Protease sequences are particularly useful as they will result in amplification of an imaging, radiotherapeutic, diagnostic, or therapeutic effect through enzymatic action on the conjugate complex, thereby increasing the intracellular concentration of a cleaved and subsequently trapped metal-chelate or other additional active agent such as an siRNA. Another suitable functional linker is a Ca-responsive protein domain such as an EF-hand domain. A Ca-responsive domain renders the complex responsive to an intracellular signaling cascade by changing conformation and activity in response to a second messenger, thereby changing activity of the complex.

In addition, the linker can be either a non-cleavable linker or a cleavable linker. The non-cleavable linker can include an amide bond or phosphate bond, and the cleavable linker can include a disulfide bond, acid-cleavable linkage, ester bond, anhydride bond, biodegradable bond, or enzyme-cleavable linkage

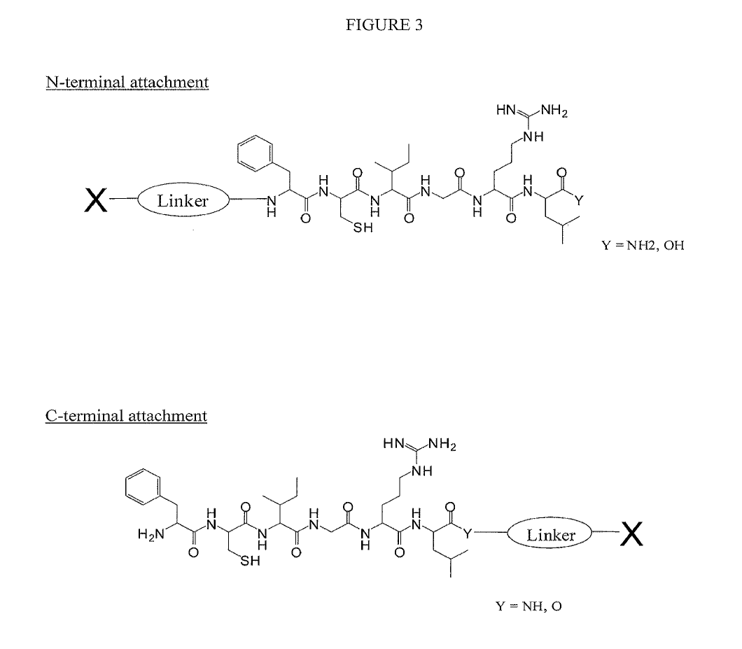

The linker may be covalently attached to the peptide permeability inducer at the N-terminus or at the C-terminus of the peptide permeability inducer sequence. In specific embodiments the peptide conjugate comprises a peptide permeability inducer consisting of the amino acid sequence of SEQ ID NO:1 (FCIGRL). In a particular embodiment the linker may be attached to the peptide permeability inducer via the N-terminal Phenylalanine residue of SEQ ID NO:1. In a particular embodiment the linker may be attached to the peptide permeability inducer via the C-terminal Leucine residue of SEQ ID NO:1. Examples of such terminal linker attachment schemes are shown in FIG. 3.

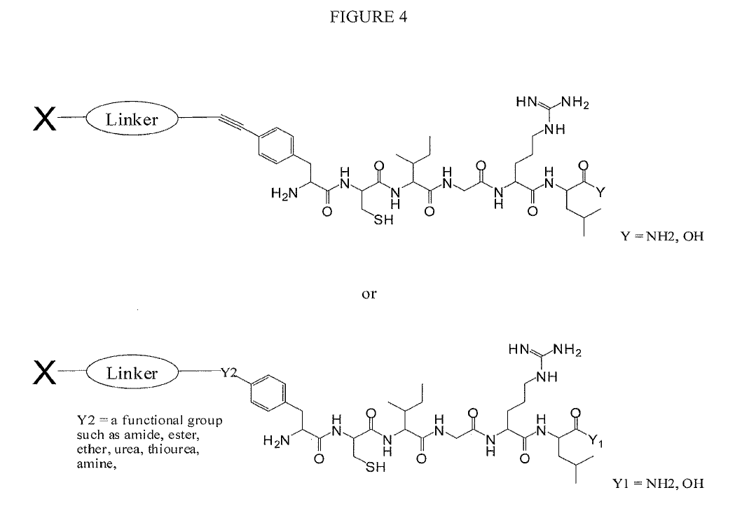

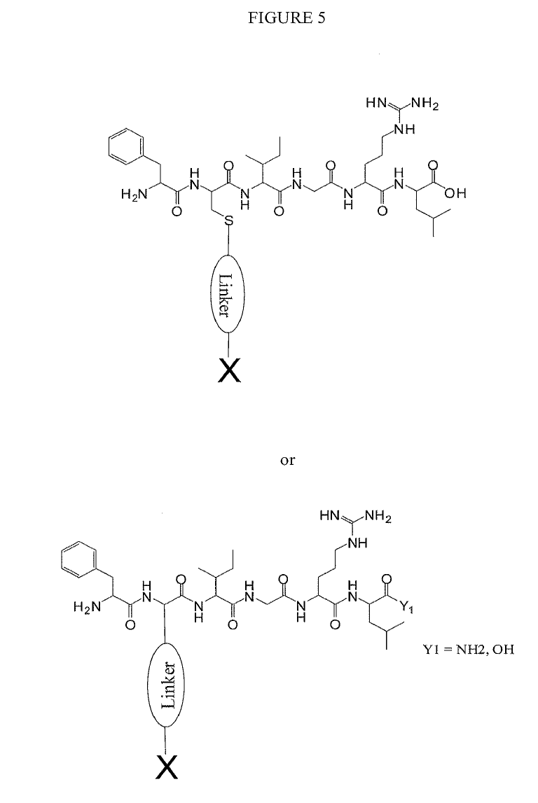

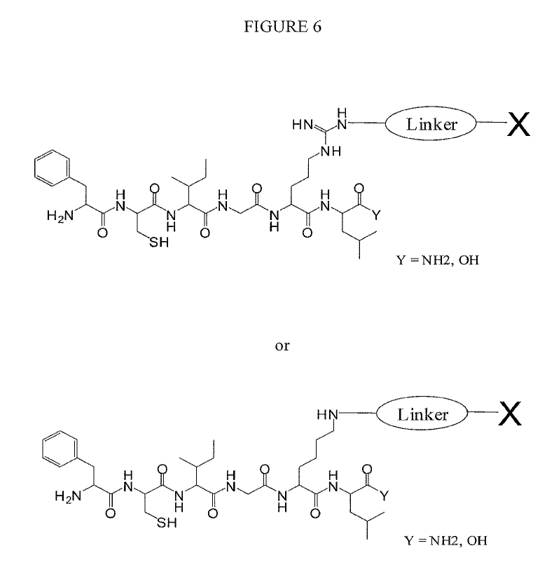

In further embodiments the linker may be attached at an amino acid side chain of the peptide permeability inducer. For example, where the peptide conjugate comprises a peptide permeability inducer consisting of the amino acid sequence of SEQ ID NO:1 (FCIGRL), the linker may be attached to the peptide permeability inducer via the side chain of the Cysteine (C), Phenylalanine (F) or Arginine (R) residues of SEQ ID NO: 1. In a particular embodiment the linker is attached to the peptide permeability inducer via the side chain of the Cysteine residue of SEQ ID NO: 1. In a particular embodiment the linker is attached to the peptide permeability inducer via the side chain of the Phenylalanine residue of SEQ ID NO:1. In a particular embodiment the linker is attached to the peptide permeability inducer via the side chain of the Arginine residue of SEQ ID NO:1. Examples of such amino acid side chain linker attachment schemes are shown in FIGS. 4-6.

Conjugates may be synthesized using linker chemistry that can be employed using standard methods such as solution and solid phase synthesis. More specifically, peptide permeability inducers can be conjugated to compounds such as peptides, oligonucleotides, and small molecules using solid phase chemistry. Alternatively, peptide conjugates can be prepared by solution phase synthesis using well-known functionalizable electrophiles and nucleophiles. In specific embodiments the reactive groups used for conjugation of peptide permeability inducers to additional active agents include an amide group (—NH₂), a carboxyl group (—COOH), an aldehyde group (—CHO).

Linkers used in peptide conjugates of the invention comprise chemical constituents such as, for example, polyethers, hydrocarbons, aromatic and heterocyclic rings, and peptide chains. Exemplary linkers of the present invention include:

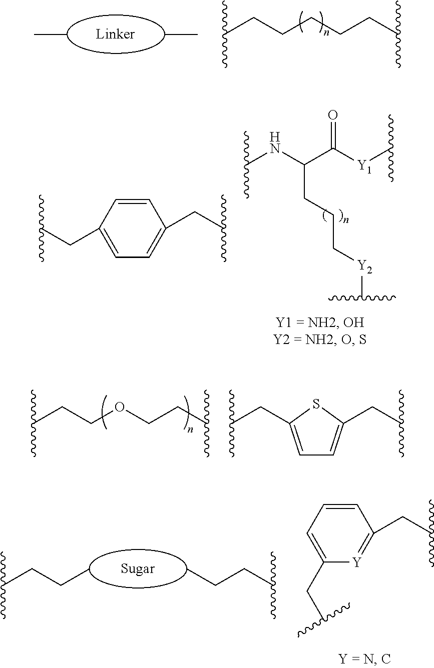

### Methods of Treatment

Peptide conjugates and pharmaceutical compositions comprising such conjugates and, optionally, one or more active agents, of the invention can be used for treating, ameliorating, and/or preventing a disease. Any disease may be treated using the compositions of the invention by selection of an appropriate active agent, e.g., therapeutic and/or immunogenic agent. In one embodiment, the present invention provides a method of treating diabetes response in a subject (e.g., a mammal such as a human) by administering a composition comprising one or more peptide conjugates together with one or more insulins and/or derivatives thereof. In some embodiments, a peptide conjugate may include an insulin. In another embodiment, the invention provides a method of suppressing an excessive or undesirable immune response in a subject (e.g., a mammal such as a human) by administering a composition comprising one or more peptide conjugates together with one or more immune-suppressive drugs that may include, for example, cyclosporin A. In some embodiments, the peptide conjugate may comprise an immune-suppresive drug, for example, cyclosporine A.

Examples of diseases that can be treated using the compositions of the invention include, but are not limited to, cancer, autoimmune diseases, vascular disease, bacterial infections, gastritis, gastric cancer, collagenous colitis, inflammatory bowel disease, osteoporosis, systemic lupus erythematosus, food allergy, asthma, and irritable bowel syndrome. For example, to treat inflammatory bowel disease, a composition comprising one or more peptide conjugates may be administered to the subject (e.g., a mammal such as a human) in need thereof.

In another example, to treat cancer of the colon or rectal area, a composition comprising a therapeutically effective amount of Erbitux® (Cetuximab) together with an absorption enhancing amount of one or more peptide conjugates may be administered to the subject (e.g., a mammal such as a human) in need thereof. In some embodiments, a peptide conjugate may comprise Cetuximab. In another example, to treat breast cancer, a composition comprising a therapeutically effective amount of Herceptin® (Trastuzumab) together with an absorption enhancing amount of one or more peptide conjugates may be administered to the subject (e.g., a mammal such as a human) in need thereof. In some embodiments, the peptide conjugate may comprise Trastuzumab. In another example, to treat various types of cancer, a composition comprising a therapeutically effective amount of Avastin® (Bevacizumab) together with an absorption enhancing amount of one or more peptide conjugates may be administered to the subject (e.g., a mammal such as a human) in need thereof. In some embodiments, the peptide conjugate may comprise Bevacizumab. Another example involves treatment of osteoporosis by administration of a composition comprising one or more peptide conjugates together with a therapeutically effective amount of Fosamax® (Alendronate) to the subject in need thereof. In some embodiments, the peptide conjugate may comprise Alendronate. Another example involves treatment of transplant rejection by administration of a composition comprising one or more peptide conjugates together with a therapeutically effective amount of Cyclosporin A to the subject in need thereof. In some embodiments, the peptide conjugate may comprise Cyclosporin A. Another example involves treatment of anemia by administration of a composition comprising one or more peptide conjugates together with a therapeutically effective amount of erythropoietin to the subject in need thereof. In some embodiments, the peptide conjugate may comprise erythropoietin. Another example involves treatment of hemophilia by administration of a composition comprising one or more peptide conjugates together with a therapeutically effective amount of Factor VIII to the subject in need thereof. In some embodiments, the peptide conjugate may comprise Factor VIII.

In some embodiments, compositions of the invention (e.g., pharmaceutical compositions) may be given repeatedly over a protracted period, i.e., may be chronically administered. Typically, compositions may be administered one or more times each day in an amount suitable to prevent, reduce the likelihood of an attack of, or reduce the severity of an attack of the underlying disease condition (e.g., diabetes, cancer, transplant rejection, etc). Such compositions may be administered chronically, for example, one or more times daily over a plurality of days.

In some embodiments, compositions of the invention (e.g., pharmaceutical compositions) may be used to treat acute attacks of the underlying disease (e.g., diabetes, cancer, transplant rejection, etc). Typically, embodiments of this type will require administration of the compositions of the invention to a subject undergoing an attack in an amount suitable to reduce the severity of the attack. One or more administrations may be used.

In some embodiments, peptide conjugates of the invention may be used in the manufacture of compositions and pharmaceutical compositions for use in the methods described above.

The following examples are provided for illustrative purposes only, and are in no way intended to limit the scope of the present invention.

### EXAMPLES

### Example 1

### Measurement of Trans Epithelial Electric Resistance (TEER) and Epithelial Flux of A Fluorescent Marker Lucifer Yellow

CaCo2 cells form monolayers that exhibit tight junctions between adjacent cells. Agonists of tight junctions can be identified by their ability to enhance the flux of compounds (e.g. ions, Lucifer Yellow) through a cell monolayer that comprises tight junctions; or by their ability to reduce TEER across a cell monolayer that comprises tight junctions. Treatment of CaCo2 monolayers with peptide FCIGRL (SEQ ID NO: 1) led to a 51-fold enhancement of Lucifer Yellow permeability through CaCo2 monolayers compared to vehicle alone. Treatment of CaCo2 monolayers with peptide FCIGRL led to a 16-fold decrease in TEER across CaCo2 monolayers compared to vehicle alone.

Tight junction agonists that can be used in peptide conjugates of the invention can be identified using the following method:

Determination of TEER and Lucifer Yellow Flux

Prepare Modified Hank's Balanced Salt Solution (MHBSS) by obtaining 1 L bottle of HBSS removing 10 ml of HBSS and replacing it with 10 ml HEPES buffer pH 7.0. Adjust pH to 7.4±0.1 using concentrated NaOH (10N).

Remove Caco-2 cells from incubator, grown on 12-well, 3.0 μM, polycarbonate Transwell® filters (Corning) and record passage#, date cells seeded and age in days.

Aspirate cell culture medium from both the apical (AP) and basolateral (BL) compartments, replacing with 0.5 ml and 1.5 ml of MHBSS, respectively. Incubate cells at 37° C. for 30 minutes.

Using the MilliCell®-ERS instrument (Millipore), measure and record the transepithelial electrical resistance (TEER) across each filter and record.

Aspirate solution from the apical compartment of each filter (n=3 per condition) and replace with 0.5 ml of control and test solutions containing Lucifer Yellow and test compound if appropriate.

Place all plates into incubator set at 37° C. (±0.2), 50 RPM (±5) for a total of 180 minutes.

At t=30, 60, 120 and 180 minutes, measure and record the transepithelial electrical resistance (TEER) across each filter using the MilliCell-ERS instrument.

At t=60, 120 and 180 minutes remove 100 μl from each basolateral compartment and place it in a 96-well plate for Lucifer Yellow analysis, replace with 100 μl of MHBSS.

Make a Lucifer Yellow standard curve with the following dilutions (7500 μM, 3750 μM, 750 μM, 375 μM, 75 μM, 37.5 μM, 7.5 μM, 3.75 μM, 0.75 μM) and pipette 1004 of each into a 96-well plate except for the first three standards mentioned above which require a 1:10 dilutions prior to transferring to the 96-well plate.

Harvest the remaining start solutions and what is left in each apical compartment into 1.5 ml vials. Freeze at −20° C. for future analysis.

Analyze each 96-well plate in a Tecan Spectra Fluor Plus using Magellan at 485 and 535 nm.

Materials:

Cells: Caco-2 cells passage 40-60 grown on Transwell® plates for 21-28 days

Culture Medium: DMEM supplemented with 10% fetal bovine serum, 1% NEAA, 1% Penn/Strep

Buffers: Hank's Balanced Salt Solution (HBSS) without calcium and magnesium

Flasks: 100×20 mm Tissue culture dish Falcon.

Plates: 12 well polycarbonate Transwell® filters; 0.3 uM pore size

### Example 2

### Identification of Tight Junction Agonists for Use in Peptide Conjugates of the Invention Using Real-Time Cell Electronic Sensing (RT-CES)

IEC6 cells form monolayers that exhibit tight junctions between adjacent cells. Agonists of tight junctions can be identified by their ability to induce changes in morphology of cells in a monolayer of cells that comprise tight junctions. Such changes in the morphology of IEC6 cells may be measured using a Real-Time Cell Electronic Sensing protocol as described below.

Tight junction agonists can be identified using the following method an used in peptide conjugates of the invention:

Materials: cells: IEC6 passage 30-50, medium: DMEM 10% no calcium no magnesium, foetal bovine serum 0.1 unit per ml bovine insulin, buffers: phosphate buffered saline (PBS) no calcium no magnesium, trypsin: 0.25% porcine trypsin in HBSS no calcium no magnesium, flasks: 100×20 mm Tissue culture dish Falcon, plates: 16× E-Plate, machine: RT-CES™ 16× system (ACEA Biosciences, Inc., San Diego, Calif.)

Wash a 75 cm² flask of confluent IEC6 cells twice with 25 ml of PBS.

Add 2.5 ml of trypsin to the flask and place back in the incubator at 37° C.

Wash cells from the surface of the flask with 10 ml of serum containing media to quench the trypsin.

Pellet the cells by centrifugation at 1500 rpm for 5 minutes aspirate the media.

Resuspend the cell pellet in 10 ml of serum free media and centrifuge at 1500 rpm for 5 minutes aspirate the media and repeat for a total of 5 washes.

Take 100 μl of cells and mix with 100 μl of trypan blue.

Count the cells four times and use the average cell concentration.

Dilute the cells to 1×10⁶ per ml in serum free media.

Add 100 μl of serum free media to each well of the ACEA plates to be used.

Insert the ACEA plate and press scan.

Run step 1 of the program to measure background.

Add 50 μl of cells to each well of the ACEA plate tap each slide of the plate 10 times and place the cells on the bench for 15 minutes to allow the cells to settle.

Insert the ACEA plate and run the scan step to check connections and run step 2-1 and 2-2 overnight.

Step 2-1 sample every 2 minutes 30 times.

Step 2-2 sample every 15 minutes 100 times.

The cell indices should be between 6-10 after the overnight run and should have reached a plateau.

Remove 100 μl of media carefully from each well.

Make up the compounds to be tested so that 50 μl contains 2× the desired final concentration.

Add 50 μl of compounds to the designated wells.

Scan the plate to check connections.

Run steps 3-1 and 3-2

Step 3-1 sample every 2 minutes 30 times.

Step 3-2 sample every 15 minutes 100 times.

### Example 3

Assay of cytoskeletal rearrangement induced by tight junction agonists that can be used in peptide conjugates of the invention

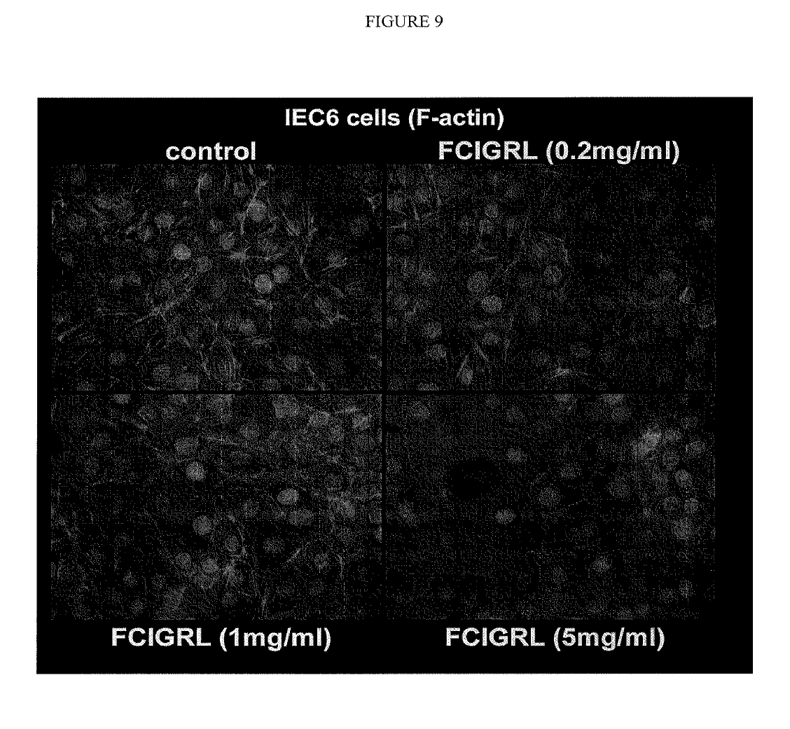

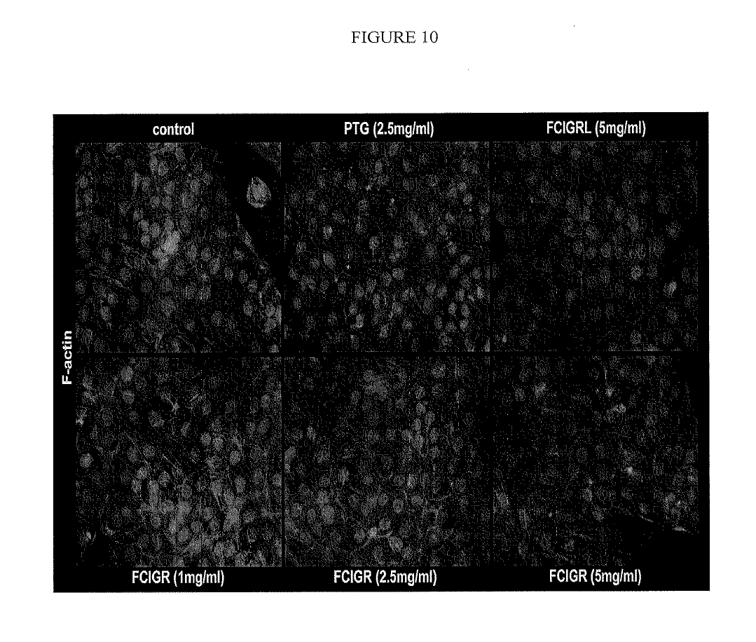

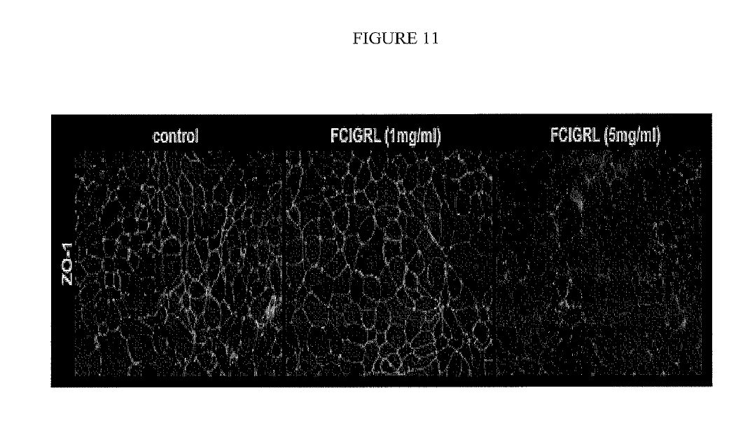

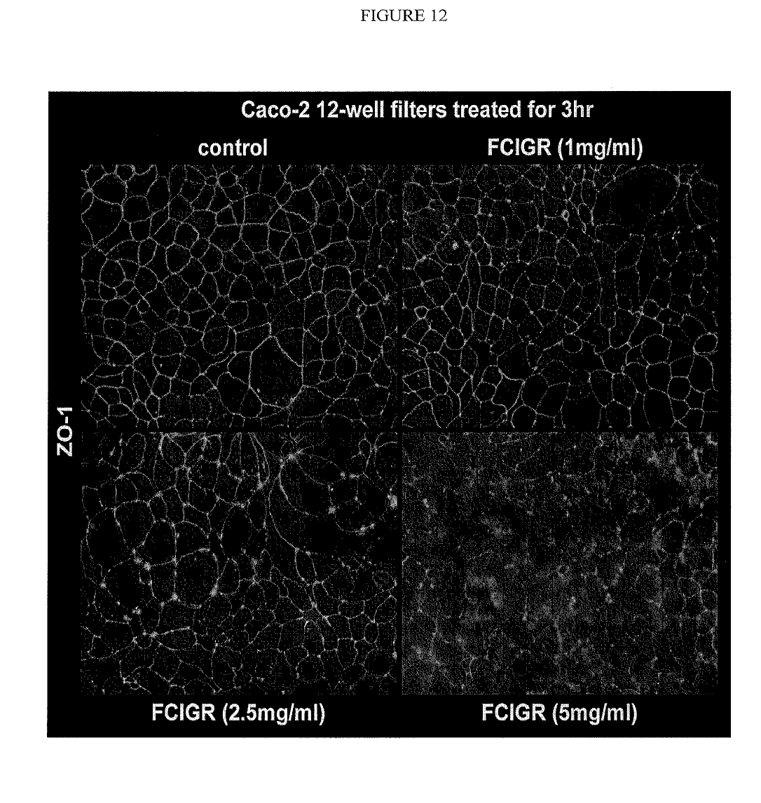

Gliadin treated with the peptidases pepsin and trypsin (termed PT-gliadin or PTG) induces a cytoskeletal arrangement in CaCo2 cells grown in monolayers. The rearrangement can be visualized using a Nikon-TE2000 epifluorescence microscope and a 40× objective and Alexa Fluor 555 conjugated phalloidin (Invitrogen, Carlsbad, Calif.), which binds specifically to F-actin. Exposure times were identical for control and agonist treated samples. The figures were generated using Adobe Photoshop CS2 v 9.0.2. The concentration of agonist was as indicated. Tight junction agonists can be identified by their ability to induce the cytoskeletal rearrangement as shown by the effects of peptide FCIGRL, a known tight junction agonist. FIGS. 9-12 show the cytoskeletal rearrangement induced by exemplary tight junction agonists of the invention.

The results of these assays are provided in the following tables. The first column of the table provides SEQ ID NO: of the peptide, the second column provides the sequence of the peptides tested, the third column provides the results of the indicated assay (i.e., ACEA, TEER reduction, or Lucifer Yellow flux).

In the following tables + indicates an enhancement of the permeability of the tight junctions were observed and − indicates no enhancement of permeability was observed.

TABLE 1
Measured activities of peptides of the invention.

| SEQ ID | | ACEA 5 | Enhanced LY | Reduced |
|---|---|---|---|---|
| NO: | Sequence | mg/mL | permeability | TEER |
| 1 | Phe-Cys-Ile-Gly-Arg-Leu | | + | + |
| 2 | Ala-Cys-Ile-Gly-Arg-Leu | + | − | − |
| 3 | Phe-Ala-Ile-Gly-Arg-Leu | − | − | − |
| 4 | Phe-Cys-Ala-Gly-Arg-Leu | NC | − | − |
| 5 | Phe-Cys-Ile-Ala-Arg-Leu | − | − | − |
| 6 | Phe-Cys-Ile-Gly-Ala-Leu | + | − | − |
| 7 | Phe-Cys-Ile-Gly-Arg-Ala | + | + | + |
| 8 | Cys-Ile-Gly-Arg-Leu | − | − | − |
| 9 | Ile-Gly-Arg-Leu | NC | − | − |
| 10 | Phe-Cys-Ile-Gly-Arg | + | + | + |
| 11 | Phe-Cys-Ile-Gly | + | | − |
| 12 | D-Phe-Cys-Ile-Gly-Arg-Leu | NC | + | + |
| 13 | Phe-D-Cys-Ile-Gly-Arg-Leu | + | + | + |
| 14 | Phe-Cys-D-Ile-Gly-Arg-Leu | − | + | + |
| 15 | Phe-Cys-Ile-Gly-D-Arg-Leu | + | − | − |
| 16 | Phe-Cys-Ile-Gly-Arg-D-Leu | − | − | − |
| 17 | D-Leu-D-Arg-Gly-D-Ile-D-Cys- | − | + | + |
| | D-Phe | | | |
| 18 | D-Leu-D-Arg-Gly-D-Ile-D-Cys- | + | + | + |
| | Phe | | | |
| 19 | D-Leu-D-Arg-Gly-D-Ile-Cys-D- | + | + | + |
| | Phe | | | |
| 20 | D-Leu-D-Arg-Gly-Ile-D-Cys-D- | ++ | + | + |
| | Phe | | | |
| 21 | D-Leu-Arg-Gly-D-Ile-D-Cys-D- | NC | − | − |
| | Phe | | | |
| 22 | Leu-D-Arg-Gly-D-Ile-D-Cys-D- | − | + | + |
| | Phe | | | |
| 23 | Leu-Arg-Gly-Ile-Cys-Phe | − | + | + |
| 24 | D-Leu-Arg-Gly-Ile-Cys-Phe | + | − | − |
| 25 | Leu-D-Arg-Gly-Ile-Cys-Phe | + | − | − |
| 26 | Leu-Arg-Gly-D-Ile-Cys-Phe | NC | + | + |
| 27 | Leu-Arg-Gly-Ile-D-Cys-Phe | NC | − | − |
| 28 | Leu-Arg-Gly-Ile-Cys-D-Phe | NC | − | − |
| 29 | D-Phe-D-Cys-D-Ile-Gly-D-Arg- | − | + | + |
| | D-Leu | | | |
| 30 | D-Phe-D-Cys-D-Ile-Gly-D-Arg- | NC | + | + |
| | Leu | | | |
| 31 | D-Phe-D-Cys-D-Ile-Gly-Arg-D- | NC | − | − |
| | Leu | | | |
| 32 | D-Phe-D-Cys-Ile-Gly-D-Arg-D- | − | + | + |
| | Leu | | | |
| 33 | D-Phe-Cys-D-Ile-Gly-D-Arg-D- | + | − | − |
| | Leu | | | |
| 34 | Phe-D-Cys-D-Ile-Gly-D-Arg-D- | NC | − | − |
| | Leu | | | |
| 35 | Phe-D-Cys-Ile-Gly-D-Arg-Leu | NC | + | + |
| 36 | D-Leu-Arg-Gly-Ile-Cys-Phe | NC | − | − |
| 37 | D-Leu-D-Arg-Gly-Ile-Cys-Phe | NC | − | − |
| 38 | D-Leu-D-Arg-Gly-Ile-Cys-D- | NC | − | − |
| | Phe | | | |
| 39 | D-Leu-D-Arg-Gly-Ile-D-Cys- | NC | − | − |
| | Phe | | | |
| 40 | Phe-Cys-Ala-Gly-Met-Ser CO2H | | | |
| 41 | Phe-Cys-Val-Gly-Met-Ser CO2H | | | |
| 42 | H-Pro-Cys-Ile-Gly-Arg-Leu-OH | | | |
| 43 | H-Gln-Cys-Ile-Gly-Arg-Leu-OH | | | |
| 44 | H-Gly-Cys-Ile-Gly-Arg-Leu-OH | | | |
| 45 | H-Thr-Cys-Ile-Gly-Arg-Leu-OH | | | |
| 46 | H-Ser-Cys-Ile-Gly-Arg-Leu-OH | | | |
| 47 | H-AsD-Cys-Ile-Gly-Arg-Leu-OH | | | |
| 48 | H-Arg-Cys-Ile-Gly-Arg-Leu-OH | | | |
| 49 | H-Val-Cys-Ile-Gly-Arg-Leu-OH | | | |
| 50 | H-Phe-Cys-Ile-Gly-Arg-Gly-OH | | | |
| 51 | H-(d)Ala-Cys-Ile-Gly-Arg- | | + | + |
| | Gly-OH | | | |
| 52 | H-Ala-Cys-Ile-Gly-Arg-Gly-OH | | | |
| 53 | H-Phe-Cys-Ile-Gly-Arg-Gly-OH | | | |
| 54 | H-(d)Phe-Cys-Ile-Gly-Arg- | | | |
| | Gly-OH | | | |
| 55 | H-Phe-Cys-Ile-Gly-Arg-Ser-OH | | | |
| 56 | H-Phe-Cys-Ile-Gly-Arg-Gln-OH | | | |
| 57 | H-Phe-Cys-Ile-Gly-Arg-Lys-OH | | | |
| 58 | H-Phe-Cys-Ile-Gly-Arg- | | | |
| | (d)Ala-OH | | | |
| 59 | H-Phe-Cys-Ile-Gly-Arg-Ile-OH | | | |
| 60 | H-Phe-Cys-Ile-Gly-Arg-Gly- | | | |
| | NH2 | | | |
| 61 | H-Phe-Cys-Ile-Gly-Arg-Asp-OH | | | |
| 62 | H-Phe-Cys-Ile-Gly-Arg-Glu-OH | | | |
| 63 | H-Phe-Cys-Ile-Gly-Arg-Phe-OH | | + | + |
| 64 | H-Phe-Cys-Ile-Gly-Arg-Asn-OH | | | |
| 65 | H-Phe-Cys-Ile-Gly-Arg-Pro-OH | | | |
| 66 | H-Glu-Cys-Ile-Gly-Arg-Leu-OH | | | |
| 67 | H-Asp-Cys-Ile-Gly-Arg-Leu-OH | | | |
| 68 | H-Lys-Cys-Ile-Gly-Arg-Leu-OH | | | |
| 69 | H-Phe-Cys-Ile-Gly-Arg-Leu- | | - | − |
| | Cys-OH | | | |
| 70 | H-Pro-Gly-Pro-Gly-Arg-Leu-OH | | | |
| 71 | H-Phe-Cys-Ile-Pro-Gly-Pro-OH | | | |
| 72 | H-Phe-Cys-Leu-Gly-Arg-Leu-OH | | | |
| 73 | H-Gly-Cys-Ile-Gly-Arg-Gly-OH | | | |
| 74 | Tyr-Cys-Ile-Gly-Arg-Leu | | + | + |
| 75 | Ac-Ala-Cys-Ile-Gly-Arg-Leu | | + | + |
| 76 | Trp-Cys-Ile-Gly-Arg-Leu | | + | + |
| 77 | Ac-Ala-Cys-Ile-Gly-Arg-Ser | | - | − |
| 78 | Ac-Ala-Cys-Ile-Gly-Arg-Ala | | + | − |
| 79 | Ac-Phe-Cys-Ile-Gly-Arg-Phe | | | |
| 80 | Ser-Leu-Ile-Gly-Arg-Leu-NH2 | | + | + |
| 81 | Phe-Cys-Ala-Gly | | | |
| 82 | Phe-Cys-Gly-Gly | | | |
| 83 | Gly-Phe-Cys-Ile-Gly-Arg-Leu | | + | + |
| 84 | Leu-Arg-Gly-Gly-Arg-Leu | | | |
| 85 | Phe-Cys-Ala-Gly-Met-Ser | | | |
| 86 | Phe-Cys-Val-Gly-Met-Ser | | | |
| 87 | Ac-Phe-Leu-Ile-Gly-Arg-Leu- | | + | + |
| | NH2 | | | |
| 88 | Tyr-Ile-Gly-Ser-Arg | | | |
| 89 | H-Sar-Cys-Ile-Gly-Arg-Leu-OH | | | |
| 90 | H-Cha-Cys-Ile-Gly-Arg-Leu-OH | | | |
| 91 | H-Aib-Cys-Ile-Gly-Arg-Leu-OH | | | |
| 92 | H-(t-Bu)Gly-Cys-Ile-Gly-Arg- | | | |
| | Leu-OH | | | |
| 93 | H-Nva-Cys-Ile-Gly-Arg-Leu-OH | | | |
| 94 | H-Hse-Cys-Ile-Gly-Arg-Leu-OH | | | |
| 95 | H-Phe-Cys-Ile-Gly-Arg-Nva-OH | | | |
| 96 | H-Phe-Cys-Ile-Gly-Arg- | | + | + |
| | betaAla-OH | | | |
| 97 | H-Phe-Cys-Ile-Gly-Arg-Tle-OH | | + | + |
| 98 | H-Phe-Cys-Ile-Gly-Arg-MeAla- | | | |
| | OH | | | |
| 99 | H-Phe-Cys-Ile-Gly-Arg-Abu-OH | | | |
| 100 | H-Phe-Cys-Ile-Gly-Arg-Aib-OH | | | |
| 101 | H-Phe-Cys-Ile-Gly-Arg-Cha-OH | | + | + |
| 102 | H-Abu-Cys-Ile-Gly-Arg-Leu-OH | | | |
| 103 | H-Orn-Cys-Ile-Gly-Arg-Leu-OH | | | |
| 104 | Phe-Cys-Ile-Gly-Cit-Leu | | - | − |
| 105 | Phe(4-NO2)-Cys-Ile-Gly-Arg- | | - | − |
| | Leu | | | |
| 106 | Phe(4-Cl)-Cys-Ile-Gly-Arg- | | | |
| | Leu | | | |
| 107 | Phe-Cys-Ile-Gly-Arg-Phe(4- | | | |
| | Cl) | | | |
| 108 | Phe-Cys-Ile-Gly-Arg-Phe(4- | | | |
| | NO2) | | | |
| 109 | Tic-Cys-Ile-Gly-Arg-Leu | | - | − |
| 110 | Phe-Cys-Ile(nMe)-Gly-Arg-Leu | | | |
| 111 | Phe-Cys-Ile-Gly-Arg-Thi | | | |
| 112 | Thi-Cys-Ile-Gly-Arg-Leu | | | |
| 113 | Phe-Cys-Ile-Gly-Arg-Tic | | | |
| 114 | H-Phe-Thr-Ile-Gly-Arg-Leu-OH | | + | + |
| 115 | H-Phe-Ser-Ile-Gly-Arg-Leu-OH | | + | + |
| 116 | H-Phe-Met-Ile-Gly-Arg-Leu-OH | | − | − |
| 117 | Phe-Leu-Ile-Gly-Arg-Leu-NH2 | | − | − |
| 118 | Phe-Phe-Leu-Ile-Gly-Arg-Leu- | | + | + |
| | NH2 | | | |
| 119 | Phe-Phe-Ile-Gly-Arg-Leu-OH | | − | + |
| 120 | Phe-Pro-Ile-Gly-Arg-Leu-OH | | − | − |
| 121 | Phe-Trp-Ile-Gly-Arg-Leu | | + | + |
| 122 | Phe-His-Ile-Gly-Arg-Leu | | | |
| 123 | Phe-Pro-Ile-Gly-Arg-Leu | | − | − |
| 124 | Phe-Asp-Ile-Gly-Arg-Leu | | − | − |
| 125 | Phe-Leu-Ile-Gly-Arg-Leu-NH2 | | + | + |
| 126 | Phe-Phe-Ile-Gly-Arg-Leu-NH2 | | + | + |
| 127 | Phe-Arg-Ile-Gly-Arg-Leu | | − | + |
| 128 | Phe-Gly-Ile-Gly-Arg-Leu | | + | − |
| 129 | Phe-Gln-Ile-Gly-Arg-Leu | | − | − |
| 130 | Phe-Glu-Ile-Gly-Arg-Leu | | − | − |
| 131 | Phe-Lys-Ile-Gly-Arg-Leu | | + | + |
| 132 | Phe-Asn-Ile-Gly-Arg-Leu | | − | + |
| 133 | Phe-Tyr-Ile-Gly-Arg-Leu | | − | − |
| 134 | Phe-Leu-Ile-Gly-Arg-Leu | | − | − |
| 135 | Phe-Val-Ile-Gly-Arg-Leu | | + | + |
| 136 | Phe-Ile-Ile-Gly-Arg-Leu | | + | + |
| 137 | Ser-Leu-Ile-Gly-Arg-Leu-CO2H | | | |
| 138 | Leu-Arg-Gly-Ile-Leu-Phe-NH2 | | + | + |
| 139 | Phe-Leu-Ile-Gly-Arg-NH2 | | − | − |
| 140 | H-Phe-Thi-Ile-Gly-Arg-Leu-OH | | − | − |
| 141 | H-Phe-Hse-Ile-Gly-Arg-Leu-OH | | + | |
| 142 | H-Phe-Abu-Ile-Gly-Arg-Leu-OH | | − | − |
| 143 | H-Phe-Met(O)-Ile-Gly-Arg- | | − | − |
| | Leu-OH | | | |
| 144 | H-Phe-Met(O)2-Ile-Gly-Arg- | | − | − |
| | Leu-OH | | | |
| 145 | H-Phe-(d)Cys-Ile-Gly-Arg- | | + | + |
| | Leu-OH | | | |
| 146 | H-Phe-Nva-Ile-Gly-Arg-Leu-OH | | − | − |
| 147 | H-Phe-Nle-Ile-Gly-Arg-Leu-OH | | − | − |
| 148 | Ac-Phe-Hse-Ile-Gly-Arg-Ala | | + | + |
| 149 | Ac-Phe-Hse-Ile-Gly-Arg-Ser | | | |
| 150 | Ac-Phe-Hse-Ile-Gly-Arg-Phe | | + | + |
| 151 | Phe-Hse-Ile-Gly-Arg-Phe | | − | − |
| 152 | Phe-Cys(S-benzyl)-Ile-Gly- | | − | − |
| | Arg-Leu | | | |
| 153 | Phe-Cys(t-buthiol)-Ile-Gly- | | − | − |
| | Arg-Leu | | | |
| 154 | Phe-Phg-Ile-Gly-Arg-Leu-OH | | + | + |
| 155 | Phe-(d)Val-Ile-Gly-Arg-Leu- | | − | − |
| | OH | | | |
| 156 | Phe-Cha-Ile-Gly-Arg-Leu-OH | | − | − |
| 157 | Phe-tBu(Gly)-Ile-Gly-Arg- | | + | + |
| | Leu-OH | | | |
| 158 | Phe-Dab-Ile-Gly-Arg-Leu | | | |
| 159 | Phe-(cyclopropane)Pro-Ile- | | − | + |
| | Gly-Arg-Leu | | | |
| 160 | Phe-Dpr-Ile-Gly-Arg-Leu | | + | + |
| 161 | Phe-Pen(Acm)-Ile-Gly-Arg-Leu | | + | + |
| 162 | Phe-Hcy-Ile-Gly-Arg-Leu | | + | + |
| 163 | Phe-(2-pyridiyl)Ala-Ile-Gly- | | − | − |
| | Arg-Leu | | | |
| 164 | Phe-Leu-(d)Ile-Gly-Arg-Leu- | | − | − |
| | NH2 | | | |
| 165 | Phe-(d)Leu-Ile-Gly-Arg-Leu- | | − | − |
| | NH2 | | | |
| 166 | Leu-Arg-Gly-(d)Ile-Leu-Phe- | | − | − |
| | NH2 | | | |
| 167 | Phe-Abu(dimer)-Ile-Gly-Arg- | | | |
| | Leu | | | |
| 168 | Phe-(Dehydro)Leu-Ile-Gly- | | + | − |
| | Arg-Leu | | | |
| 169 | Ac-Phe-Hse-Ile-Gly-Arg-NH2 | | − | − |
| 170 | Phe-Hse-(d)Ile-Gly-Arg-NH2 | | − | − |
| 171 | Ac-Phe-Hse-Ile-Gly-Arg-Leu- | | − | − |
| | NH2 | | | |
| 172 | Phe-Hse-(d)Ile-Gly-Arg-Leu | | − | − |
| 173 | Phe-(4-CN)-Phe-Ile-Gly-Arg- | | − | − |
| | Leu | | | |
| 174 | Phe-(3-Me)-Phe-Ile-Gly-Arg- | | − | − |
| | Leu | | | |
| 175 | Phe-Cyclopropyl(Ala)-Ile- | | − | − |
| | Gly-Arg-Leu | | | |
| 176 | Phe-Pra-Ile-Gly-Arg-NH2 | | − | − |
| 177 | Phe-(2-furyl)Ala-Ile-Gly- | | | |
| | Arg-NH2 | | | |
| 178 | Phe-Thh-Ile-Gly-Arg-NH2 | | | |
| 179 | Phe-StyrylGly-Ile-Gly-Arg- | | | |
| | Leu-NH2 | | | |
| 180 | Phe-HOCit-Ile-Gly-Arg-Leu- | | | |
| | NH2 | | | |
| 181 | Phe-Thh-Ile-Gly-Arg-Leu-NH2 | | | |
| 182 | Phe-(2-furyl)Ala-Ile-Gly- | | | |
| | Arg-Leu-NH2 | | | |
| 183 | Phe-AllylGly-Ile-Gly-Arg- | | + | + |
| | Leu-OH | | | |
| 184 | Phe-AllylGly-Ile-Gly-Arg-NH2 | | − | − |
| 185 | Phe-(d)AllylGly-Ile-Gly-Arg- | | − | − |
| | NH2 | | | |
| 186 | Met-AllylGly-Ile-Gly-Arg- | | − | − |
| | Leu-NH2 | | | |
| 187 | Gln-AllylGly-Ile-Gly-Arg- | | − | − |
| | Leu-NH2 | | | |
| 188 | Leu-AllylGly-Ile-Gly-Arg- | | − | − |
| | Leu-NH2 | | | |
| 189 | Ser-AllylGly-Ile-Gly-Arg- | | − | − |
| | Leu-NH2 | | | |
| 190 | Thr-AllylGly-Ile-Gly-Arg- | | − | − |
| | Leu-NH2 | | | |
| 191 | Glu-AllylGly-Ile-Gly-Arg- | | − | − |
| | Leu-NH2 | | | |
| 192 | Val-AllylGly-Ile-Gly-Arg- | | − | − |
| | Leu-NH2 | | | |
| 193 | Tyr-AllylGly-Ile-Gly-Arg- | | − | − |
| | Leu-NH2 | | | |
| 194 | Gly-AllylGly-Ile-Gly-Arg- | | − | − |
| | Leu-NH2 | | | |
| 195 | Asp-AllylGly-Ile-Gly-Arg- | | − | − |
| | Leu-NH2 | | | |
| 196 | Trp-AllylGly-Ile-Gly-Arg- | | + | − |
| | Leu-NH2 | | | |
| 197 | Lys-AllylGly-Ile-Gly-Arg- | | − | − |
| | Leu-NH2 | | | |
| 198 | Ala-AllylGly-Ile-Gly-Arg- | | − | − |
| | Leu-NH2 | | | |
| 199 | His-AllylGly-Ile-Gly-Arg- | | − | − |
| | Leu-NH2 | | | |
| 200 | Pro-AllylGly-Ile-Gly-Arg- | | − | − |
| | Leu-NH2 | | | |
| 201 | Arg-AllylGly-Ile-Gly-Arg- | | − | − |
| | Leu-NH2 | | | |
| 202 | Ile-AllylGly-Ile-Gly-Arg- | | − | − |
| | Leu-NH2 | | | |
| 203 | Phe-AllylGly-Pro-Gly-Arg- | | − | − |
| | Leu-NH2 | | | |
| 204 | Phe-AllylGly-Phe-Gly-Arg- | | + | = |
| | Leu-NH2 | | | |
| 205 | Phe-AllylGly-Thr-Gly-Arg- | | − | − |
| | Leu-NH2 | | | |
| 206 | Phe-AllylGly-Leu-Gly-Arg- | | + | + |
| | Leu-NH2 | | | |
| 207 | Phe-AllylGly-Ser-Gly-Arg- | | + | + |
| | Leu-NH2 | | | |
| 208 | Phe-AllylGly-Phe-Gly-Arg- | | + | + |
| | Leu-NH2 | | | |
| 209 | Phe-AllylGly-Val-Gly-Arg- | | − | − |
| | Leu-NH2 | | | |
| 210 | Phe-AllylGly-Gly-Gly-Arg- | | + | + |
| | Leu-NH2 | | | |
| 211 | Phe-AllylGly-Ala-Gly-Arg- | | + | + |
| | Leu-NH2 | | | |
| 212 | Phe-AllylGly-His-Gly-Arg- | | | |
| | Leu-NH2 | | | |
| 213 | Phe-AllylGly-Asp-Gly-Arg- | | | |
| | Leu-NH2 | | | |
| 214 | Phe-AllylGly-Glu-Gly-Arg- | | | |
| | Leu-NH2 | | | |
| 215 | Phe-AllylGly-Gln-Gly-Arg- | | | |
| | Leu-NH2 | | | |
| 216 | Phe-AllylGly-Arg-Gly-Arg- | | | |
| | Leu-NH2 | | | |
| 217 | Phe-AllylGly-Lys-Gly-Arg- | | | |
| | Leu-NH2 | | | |
| 218 | Phe-AllylGly-Asn-Gly-Arg- | | | |
| | Leu-NH2 | | | |
| 219 | Phe-AllylGly-Tyr-Gly-Arg- | | | |
| | Leu-NH2 | | | |
| 220 | Phe-AllylGly-Ile-Thr-Arg- | | − | − |
| | Leu-NH2 | | | |
| 221 | Phe-AllylGly-Ile-Leu-Arg- | | | |
| | Leu-NH2 | | | |
| 222 | Phe-AllylGly-Ile-Ile-Arg- | | | |
| | Leu-NH2 | | | |
| 223 | Phe-AllylGly-Ile-Ala-Arg- | | + | + |
| | Leu-NH2 | | | |
| 224 | Phe-AllylGly-Ile-Pro-Arg- | | − | − |
| | Leu-NH2 | | | |
| 225 | Phe-AllylGly-Ile-Gly-Arg- | | | |
| | Leu-NH2 | | | |
| 226 | Phe-AllylGly-Ile-His-Arg- | | | |
| | Leu-NH2 | | | |
| 227 | Phe-AllylGly-Ile-Asp-Arg- | | | |
| | Leu-NH2 | | | |
| 228 | Phe-AllylGly-Ile-Glu-Arg- | | | |
| | Leu-NH2 | | | |
| 229 | Phe-AllylGly-Ile-GlD-Arg- | | | |
| | Leu-NH2 | | | |
| 230 | Phe-AllylGly-Ile-Phe-Arg- | | | |
| | Leu-NH2 | | | |
| 231 | Phe-AllylGly-Ile-Arg-Arg- | | | |
| | Leu-NH2 | | | |
| 232 | Phe-AllylGly-Ile-Lys-Arg- | | | |
| | Leu-NH2 | | | |
| 233 | Phe-AllylGly-Ile-Asn-Arg- | | | |
| | Leu-NH2 | | | |
| 234 | Phe-AllylGly-Ile-Ser-Arg- | | | |
| | Leu-NH2 | | | |
| 235 | Phe-AllylGly-Ile-Val-Arg- | | | |
| | Leu-NH2 | | | |
| 236 | Phe-AllylGly-Ile-Gly-His- | | | |
| | Leu-NH2 | | | |
| 237 | Phe-AllylGly-Ile-Gly-Asp- | | | |
| | Leu-NH2 | | | |
| 238 | Phe-AllylGly-Ile-Gly-Glu- | | | |
| | Leu-NH2 | | | |
| 239 | Phe-AllylGly-Ile-Gly-Gln- | | | |
| | Leu-NH2 | | | |
| 240 | Phe-AllylGly-Ile-Gly-Gly- | | | |
| | Leu-NH2 | | | |
| 241 | Phe-AllylGly-Ile-Gly-Ala- | | | |
| | Leu-NH2 | | | |
| 242 | Phe-AllylGly-Ile-Gly-Phe- | | | |
| | Leu-NH2 | | | |
| 243 | Phe-AllylGly-Ile-Gly-Lys- | | | |
| | Leu-NH2 | | | |
| 244 | Phe-AllylGly-Ile-Gly-Leu- | | | |
| | Leu-NH2 | | | |
| 245 | Phe-AllylGly-Ile-Gly-Met- | | | |
| | Leu-NH2 | | | |
| 246 | Phe-AllylGly-Ile-Gly-Asn- | | | |
| | Leu-NH2 | | | |
| 247 | Phe-AllylGly-Ile-Gly-Ser- | | | |
| | Leu-NH2 | | | |
| 248 | Phe-AllylGly-Ile-Gly-Tyr- | | | |
| | Leu-NH2 | | | |
| 249 | Phe-AllylGly-Ile-Gly-Thr- | | | |
| | Leu-NH2 | | | |
| 250 | Phe-AllylGly-Ile-Gly-Ile- | | | |
| | Leu-NH2 | | | |
| 251 | Phe-AllylGly-Ile-Gly-Trp- | | | |
| | Leu-NH2 | | | |
| 252 | Phe-AllylGly-Ile-Gly-Pro- | | | |
| | Leu-NH2 | | | |
| 253 | Phe-AllylGly-Ile-Gly-Val- | | | |
| | Leu-NH2 | | | |
| 254 | Phe-AllylGly-Ile-Gly-Arg- | | | |
| | His-NH2 | | | |
| 255 | Phe-AllylGly-Ile-Gly-Arg- | | | |
| | Asp-NH2 | | | |
| 256 | Phe-AllylGly-Ile-Gly-Arg- | | | |
| | Arg-NH2 | | | |
| 257 | Phe-AllylGly-Ile-Gly-Arg- | | | |
| | Phe-NH2 | | | |
| 258 | Phe-AllylGly-Ile-Gly-Arg- | | | |
| | Ala-NH2 | | | |
| 259 | Phe-AllylGly-Ile-Gly-Arg- | | | |
| | Gly-NH2 | | | |
| 260 | Phe-AllylGly-Ile-Gly-Arg- | | | |
| | Gly-NH2 | | | |
| 261 | Phe-AllylGly-Ile-Gly-Arg- | | | |
| | Glu-NH2 | | | |
| 262 | Phe-AllylGly-Ile-Gly-Arg- | | | |
| | Thr-NH2 | | | |
| 263 | Phe-AllylGly-Ile-Gly-Arg- | | | |
| | Tyr-NH2 | | | |
| 264 | Phe-AllylGly-Ile-Gly-Arg- | | | |
| | Ser-NH2 | | | |
| 265 | Phe-AllylGly-Ile-Gly-Arg- | | | |
| | Asn-NH2 | | | |
| 266 | Phe-AllylGly-Ile-Gly-Arg- | | | |
| | Met-NH2 | | | |
| 277 | Phe-AllylGly-Ile-Gly-Arg- | | | |
| | Lys-NH2 | | | |
| 288 | Phe-AllylGly-Ile-Gly-Arg- | | | |
| | Ile-NH2 | | | |
| 289 | Phe-AllylGly-Ile-Gly-Arg- | | | |
| | Trp-NH2 | | | |
| 290 | Phe-AllylGly-Ile-Gly-Arg- | | | |
| | Pro-NH2 | | | |
| 291 | Phe-Ally1Gly-Ile-Gly-Arg- | | | |
| | Val-NH2 | | | |

TABLE 2
Abbreviations and Structures of Non-naturally Occurring Amino Acids.

| Abbreviation | IUPAC Name | Chemical Structure |
|---|---|---|
| Sar | Sarcosine | 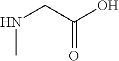 |
| Aib | alpha- aminoisobutyric acid | 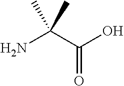 |
| Tle | L-tert-Leucine | 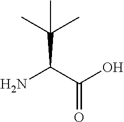 |
| MeAla | L-N-methyl Alanine | 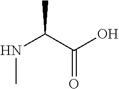 |
| Abu | L-alpha- aminobutyric acid | 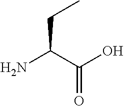 |
| Phe(4-NO₂) | L-4-nitro- phenylalanine | 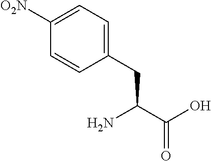 |
| Phe(4-Cl) | L-4- chlorophenylalanine | 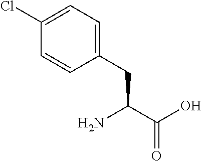 |
| Tic | L-1,2,3,4- tetrahydro- isoquinoline- 3-carboxylic acid | 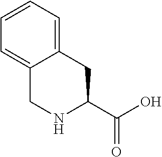 |
| Thi | L-beta-(2-thienyl)- alanine | 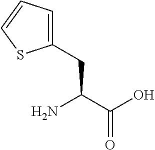 |
| Met(O) | L-methionine- sulfoxide | 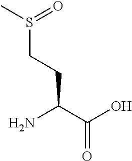 |
| Met(O)₂ | L-methionine- sulfone | 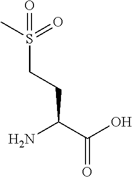 |
| Nle | L-norleucine | 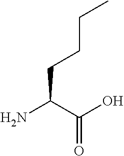 |
| Cys(S-benzyl) | L-S-benzyl- cysteine | 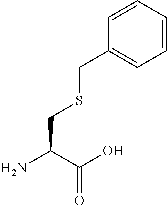 |
| Cys(t-buthiol) | L-S-tert-butylthio- cysteine | 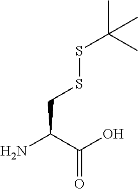 |
| Phg | L-phenylglycine | 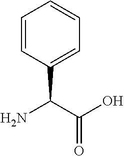 |
| Cha | L-beta-cyclohexyl- alanine | 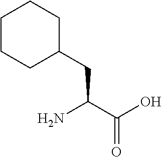 |
| (Allyl)Gly | L-allylglycine | 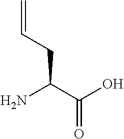 |
| (t-Bu)Gly | L-tert-butylglycine | 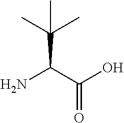 |
| Dab | L-1,4- diaminobutyric acid | 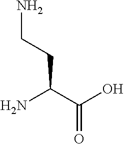 |
| (cyclopropane) Pro | L-(R,S)-3,4- cis-methanoproline | 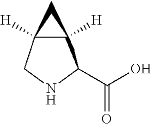 |
| Dap/Dpr | L-1,3- diaminopropionic acid | 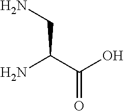 |
| Pen(Acm) | L-S- acetamidomethyl- penicillamine | 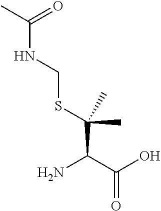 |
| (2-pyridyl)Ala | L-beta-(2-pyridyl)- alanine | 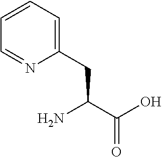 |
| (4,5-dehydro) Leu | L-4,5-dehydro- leucine | 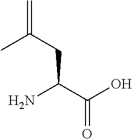 |
| Phe(4-CN) | L-4-cyano- phenylalanine | 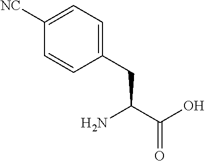 |
| Phe(3-Me) | L-3-methyl- phenylalanine | 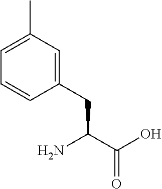 |
| (cyclopropyl) Ala | L-beta-cyclopropyl- alanine | 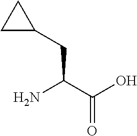 |
| Pra | L-propargylglycine | 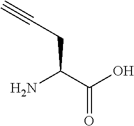 |
| (2-furyl)Ala | L-beta-(2-furyl)- alanine | 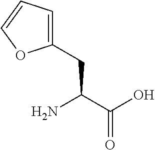 |
| Thh | 1,2,3,4- tetrahydroharmane- 3-carboxylic acid | 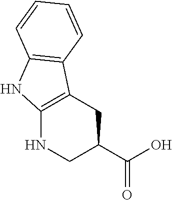 |
| (styryl)Gly | L-beta-styryl- alanine | 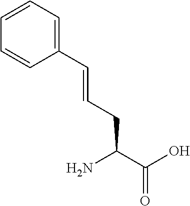 |
| HOCit | Homocitrulline | 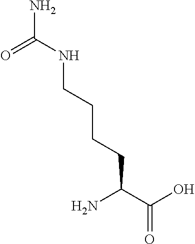 |

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A peptide conjugate comprising a peptide comprising of an amino acid sequence selected from the group of SEQ ID NOs: 1-291, a linker, and an additional active agent.

2. The peptide conjugate of claim 1, wherein the peptide is three to ten amino acids in length.

3. The peptide conjugate of claim 1, wherein the linker is selected form the group consisting of: a functional linker and a non-functional linker.

4. The peptide conjugate of claim 1, wherein the linker is covalently attached to the peptide at a site selected from the group consisting of: the N-terminus, the C-terminus, and an amino acid side chain.

5. A pharmaceutical composition comprising the peptide conjugate of claim 1 and a pharmaceutically acceptable excipient.

6. A method of treating a disease in a subject in need thereof, comprising: administering to said subject the peptide conjugate of claim 1.

7. The method of claim 6, wherein the disease is selected from the group consisting of diabetes, an excessive or undesirable immune response, inflammatory bowel disease, and cancer.

8. The peptide conjugate of claim 1, wherein the peptide comprises a sequence selected from the group consisting of SEQ ID NOs:1, 2, 6, 7, 10, 11, 12, 13, 14, 17, 18, 19, 20, 22, 23, 24, 25, 26, 29, 30, 32, 35, 51, 63, 74, 75, 76, 78, 80, 83, 87, 96, 97, 101, 114, 115, 118, 119, 121, 125, 126, 127, 131, 132, 135, 136, 138, 141, 145, 148, 150, 154, 157, 159, 160, 161, 162, 168. 183, 196, 204, 206, 207, 208, 210, 211 and 223.

9. The peptide conjugate of claim 8, wherein the linker is selected form the group consisting of: a functional linker and a non-functional linker.

10. The peptide conjugate of claim 8, wherein the linker is covalently attached to the peptide at a site selected from the group consisting of: the N-terminus, the C-terminus, and an amino acid side chain.

11. A pharmaceutical composition comprising the peptide conjugate of claim 8 and a pharmaceutically acceptable excipient.

12. A method of treating a disease in a subject in need thereof, comprising: administering to said subject the peptide conjugate of claim 8.

13. The method of claim 12, wherein the disease is selected from the group consisting of diabetes, an excessive or undesirable immune response, inflammatory bowel disease, and cancer.

14. The peptide conjugate of claim 1, wherein the additional active agent is selected from the group consisting of therapeutic agents, imaging agents, and immunogenic agents.

15. The peptide conjugate of claim 14, wherein the additional active agent is a therapeutic agent selected from the group consisting of glucose metabolism agents, antibiotics, antineoplastics, antihypertensives, antiepileptics, central nervous system agents, anti-inflammatory agents and immune system suppressants.

16. The peptide conjugate of claim 14, wherein the linker is selected form the group consisting of: a functional linker and a non-functional linker.

17. The peptide conjugate of claim 14, wherein the linker is covalently attached to the peptide at a site selected from the group consisting of: the N-terminus, the C-terminus, and an amino acid side chain.

18. A pharmaceutical composition comprising the peptide conjugate of claim 14 and a pharmaceutically acceptable excipient.

19. A method of treating a disease in a subject in need thereof, comprising: administering to said subject the peptide conjugate of claim 14.

20. The method of claim 19, wherein the disease is selected from the group consisting of diabetes, an excessive or undesirable immune response, inflammatory bowel disease, and cancer.

21. The peptide conjugate of claim 1, wherein the additional active agent is selected from the group consisting of a nucleic acid, a polypeptide and a small molecule.

22. The peptide conjugate of claim 21, wherein the linker is selected form the group consisting of: a functional linker and a non-functional linker.

23. The peptide conjugate of claim 21, wherein the linker is covalently attached to the peptide at a site selected from the group consisting of: the N-terminus, the C-terminus, and an amino acid side chain.

24. The peptide conjugate of claim 21, wherein the additional active agent is a nucleic acid selected from the group consisting of: a deoxyribonucleic acid (DNA), a ribonucleic acid (RNA), a short interfering nucleic acid (siNA), a short interfering RNA (siRNA), a double-stranded RNA (dsRNA), a micro-RNA (miRNA), and a short hairpin RNA (shRNA).

25. The peptide conjugate of claim 21, wherein the additional active agent is a polypeptide selected from the group consisting of: an immunoglobulin (Ig), a scFv, a growth factor, a cytokine, a chemokine, and an interleukin.

26. The peptide conjugate of claim 21, wherein the additional active agent is a small molecule selected from the group consisting of: an immunosuppressant, a chemotherapeutic, and an anti-inflammatory.

27. A pharmaceutical composition comprising the peptide conjugate of claim 21 and a pharmaceutically acceptable excipient.

28. A method of treating a disease in a subject in need thereof, comprising: administering to said subject the peptide conjugate of claim 21.

29. The method of claim 28, wherein the disease is selected from the group consisting of diabetes, an excessive or undesirable immune response, inflammatory bowel disease, and cancer.
